(19) United States
(12) Patent Application Publication
(10) Pub. No.: US 2022/0378807 A1
(43) Pub. Date: Dec. 1, 2022

(54) DELIVERY OF A COOLING AGENT TO THE PHARYNGEAL-ESOPHAGEAL SURFACE

(71) Applicant: Edward Tak Wei, Berkeley, CA (US)

(72) Inventor: Edward Tak Wei, Berkeley, CA (US)

(21) Appl. No.: 17/803,407

(22) Filed: Jun. 23, 2022

Related U.S. Application Data
(63) Continuation-in-part of application No. 16/601,056, filed on Oct. 14, 2019.

(51) Int. Cl. A61K 31/66; A61K 47/42; A61K 47/10; A61K 9/06; A61K 9/00; A61P 29/02

(52) U.S. Cl. CPC A61K 31/66; A61K 47/42; A61K 47/10; A61K 9/06; A61K 9/0053; A61P 29/02

(57) ABSTRACT

The present discovery generally pertains to the formulation of therapeutic compounds to treat the symptoms of esophageal disorders. More specifically, the present discovery pertains to two 1-di-alkyl-phosphinoyl-alkanes (DIPA) called DIPA-1-8 and DIPA-1-9. These compounds are formulated as a shaped medicament and swallowed to suppress the symptoms of esophageal reflux and dyspepsia. The DIPA act by creating sensations of coolness and cold on the pharyngeal and esophageal lining. Some of the symptoms relieved include cough, chronic cough, heartburn, chest pain, bloat, belching, and dyspepsia. A preferred embodiment is DIPA-1-9 dissolved in a gel matrix. An aspect of the invention is to design the medicament to be intercepted, impeded, ensnarled, or trapped in the pharyngeal valleculae and pyriform sinuses before it passes down the esophagus. The goal is to prolong the transit time of the medicament, also herein sometimes call the Shaped-Gel, in the hypopharynx and esophagus, so the active ingredient has ample time to dissolve in saliva and reach receptors for cooling. By experiment, the ideal formulation of the Shaped-Gel was a flat rectangular or toroid shape, with a mass of 0.3 to 0.8 g. Flatness was defined as a pill with the shortest axis, preferentially 5 to 45% of the longest axis.

### CROSS-REFERENCE TO RELATED APPLICATIONS

This application is a continuation-in-part of U.S. application Ser. No. 16/601,056, filed on Feb. 14, 2019, which has received a Notice of Allowance on Apr. 15, 2022.

### BACKGROUND OF THE INVENTION

A drug is defined as “First-in-Class” when it uses a new and unique mechanism of action for treating a medical condition. The first-in-class designation is one indicator of the innovative nature of a drug. For a molecule to succeed, it is necessary to choose the proper mechanism of action, the suitable molecule, the target site for delivery, and a system to deliver the correct dose at the right time. If any of these parameters fail, the drug will not work. Currently, no topical medication targets esophageal nerve endings to treat esophagitis, heartburn, indigestion, and chest pain symptoms.

### Drug Targets and Delivery to the Esophageal Lumen

The lumen of the mouth, pharynx, and esophagus is a conduit for food and liquid. On an average day for an adult, this foodway contacts 2 L of fluids, 1 L of saliva, and 2 kg of food. Traffic flow in the foodway synchronizes to ensure that food and liquids travel down the esophagus and not into the airway. The efficiency of this system is visible and self-evident, for example, when a large pizza is consumed with a drink. The masticated bolus transit from mouth to stomach occurs with a minimum of fuss and occurs in about one second. When the bolus is pushed into the oropharynx and swallowed, the epiglottis, like a trapdoor, drops over the glottis. The oropharynx and hypopharynx is continuous in histology but anatomically demarcated at the level of the cricoid cartilage. The hypopharynx connects to the proximal end of the esophagus at the upper esophageal sphincter. The distal segment of the esophagus terminates at the lower esophageal sphincter, at its junction with the stomach.

The lumenal surfaces of the pharynx and esophagus contact physical, chemical, and biological agents such as refluxed digestive enzymes and acid, infectious agents such as viruses, and mediators of the immune response. Symptoms of inflammation in this foodway manifest as dysphagia, indigestion (dyspepsia), satiety, nausea, globus, epigastric pain, heartburn, chest pain, regurgitation, a sour taste in the mouth, eructation (belching), and sometimes halitosis and hiccups. These foodway symptoms feel different from irritation of other body parts, such as the skin. The receptive fields of nerve endings for noxious signals are in spinal afferents and the afferents of the 9^{th}, 10^{th} cranial nerves. These nerves also convey signals of thermosensation, such as cooling.

Menthol lozenges and pastilles are over-the-counter items for alleviating sore or irritated throats, cough, and in some instances, satiety. The Halls menthol lozenge for cough was introduced in the United Kingdom over ninety years ago and continues to sell. Menthol lozenges typically weigh about 3.4 g (Walgreens cough drops) or 2.7 g (N'Ice lozenges) and contain from 5 to 10 mg of (−)-menthol in a sugar-dye matrix. Doses of menthol higher than 7 mg do not sell well because of their harsh taste. Also, when high doses of menthol enter the esophagus, an unpleasant cold is felt behind the sternum. The menthol lozenge is held in the mouth for about 10 to 15 min and dissolves in saliva before it becomes active. The menthol lozenge or pastille usually has a sweetening agent. The overall effects of the menthol lozenge on cough are complex because the mechanical presence of the lozenge inhibits swallowing.

About four decades ago, Watson et al. of Wilkinson Sword (WS) synthesized over 1200 compounds to find cooling agents that had properties better than menthol [New compounds with the menthol cooling effect. J. Soc. Cosmet. Chem. 29: 185-200, 1978]. From this research, N-alkyl-cycloalkyl- and an N-alkyl-alkyl carboxamide, WS-3, WS-5, WS-12, and WS-23, were identified and are used today as additives for confectionery, comestibles, (e.g., chewing gum), toothpaste and toiletries. None of these menthol analogs are used for medical treatment, although the idea of using a cooling agent for heartburn was considered (Zanone, U.S. Pat. No. 6,497,859; Bancovin et al. The infusion of menthol into the esophagus evokes cold sensations in healthy subjects but induces heartburn in patients with gastroesophageal reflux disease (GERD). Dis. Esophagus. 2019; 32(11):1-6).

The formidable technical challenges of topical drug delivery to the esophagus were discussed for treating eosinophilic esophagitis (EE), an immune disorder that affects children [Hirano et al., Drug Development for Eosinophilic Esophagitis. Clin Gastroenterol Hepatol. 2017. 15: 1173-1183. doi:10.1016/j.cgh.2017.03.016; Krause et al., The EsoCap-system. An innovative platform to drug targeting in the esophagus.J Control Release 2020:327:1-7. doi.org/10.1016/j.jconrel.2020.08.011]. Proton-pump inhibitors (PPI), the standard treatment for acid reflux disorders, do not work for EE, so the alternative is to deliver anti-inflammatory steroids by topical administration. However, the fast transit time of the bolus and the water-insoluble characteristics of steroids hinder the effective formulation of the drug candidates.

### BRIEF SUMMARY OF THE INVENTION

In this discovery, the goal is to apply a cooling agent to nerve endings of the hypopharynx and upper esophagus to treat esophageal irritation. This drug design strategy is not described in the prior art. In earlier studies, we used liquid drops applied to the oropharynx to control cough and pharyngeal discomfort. Here, the hypopharynx-esophageal drug target is further down the throat. The cooling agent should avoid the mouth and have time to reach its receptor in the esophagus before exiting into the stomach. The delivery method of the cooling agent to the esophageal target must overcome the short pharyngeal transit time (PTT) of 1 sec for a swallowed bolus.

In one aspect of the present invention a therapeutic method is described to treat symptoms of an esosphagus disorder whereby a medicament herein often referred to as a Shaped-Gel is orally administered in a gelatin-glycerol-water matrix. The Shaped-Gel contains a cooling agent which is a 1-dialkyl-phosphinoyl-alkane, preferably 1-Diisopropyl-phosphinoyl-octane or 1-Diisopropyl-phosphinoyl-nonane. Thus, in another aspect a Shaped-Gel is provided for oral administration of a therapeutically effective amount of a 1-[diisopropy-phosphinoy-alkane and is adapted to delay transit time in the esophagus, such as by the Shaped-Gel having a flat shape with a shortest axis that is 5% to 35% of its longest axis.

The formulation of the active ingredient in a gelatin, glycerol, and water matrix is such that it will quickly dissolve in body fluids such as saliva. The rapid dissolution ensures safety because even if the gel enters the airways it will dissolve and not obstruct. Thus, unintended entry of the formulation into the airways is not a hazard because it will not leave residues. This gel is here called a “Shaped-Gel,” and its size and shape are configured to be intercepted, impeded, ensnarled, or trapped in the pharyngeal valleculae and pyriform sinuses before it passes down the esophagus. The non-standard shape prolongs transit time of the Shaped-Gel and allows the active ingredient greater contact time with its receptor.

The pharynx, commonly called the throat, is a funnel-shaped passage that connects the mouth and nose to the esophagus. The pharynx subdivides into the nasopharynx, oropharynx, and hypopharynx (also called the laryngopharynx). The hypopharynx and esophagus occupy a central position in the neck and chest. At the lower border of the oropharynx are structures called valleculae. The epiglottic vallecula are indented spaces, separated by the median glossoepiglottic fold, at the root of the tongue, behind the anterior surface of the epiglottis. The oropharynx merges into the hypopharynx which connects to the esophagus, a muscular tube about eight to ten inches (25 cm) long that connects to the stomach. At the side of the voicebox (larynx) and within the hypopharynx are the pyriform sinuses. The valleculae and pyriform sinuses are indentations of space where the Shaped-Gel can be trapped and not immediately move into the esophagus. The esophagus begins behind the windpipe (trachea), in front of the spine, and in the center of the neck. At the entrance of the esophagus is the upper esophageal sphincter, which is usually closed like a clenched fist. The sphincter opens when a bolus of food, liquid, or topical medication is swallowed, and the head of the bolus reaches the sphincter. The bolus moves quickly, and the pharyngeal transit time (PTT) is 1 sec.

When the luminal esophageal surface is injured or inflamed, pain and discomfort occur locally and may irradiate to adjacent structures such as the trachea and the cardia. Typical symptoms of esophageal disorders such as reflux disease, caused by the excess acid or digestive juices, are heartburn, regurgitation, and chest pain. Other symptoms are fullness, chest discomfort, early satiation, bloating, belching, nausea, vomiting, or pain. Current treatment mainly manages acidity, e.g., with proton-pump inhibitors (PPI), H₂-receptor antagonists such as famotidine, and antacids. In laryngeal reflux, gastric contents are aerosolized up the esophagus and pharynx, as shown by the sophisticated technique of scintigraphy using radioactive technetium [Park et al. Modified Reflux Scintigraphy Detects Pulmonary Microaspiration in Severe Gastro-Esophageal and Laryngopharyngeal Reflux Disease. Lung [Internet]. https://doi.org/10.1007/s00408-021-00432-y Dyspepsia, or indigestion, is a condition of similar symptoms and etiology.

The choice of a cooling molecule is for a particular compound within the series of compounds known as phosphine oxides (which have the following general formula), and more particularly, an example of the group known as di-alkyl-phosphinoyl-alkanes (herein referred to as “DAPA compounds”) (wherein each of R₁, R₂, and R₃ is an alkyl group). And more specifically, to one particular 1-diisopropyl-phosphinoyl-alkane (DIPA), 1-Diisopropyl-phosphinoyl-nonane, referred to herein as “DIPA-1-9”. In previous studies (U.S. Ser. No. 16/501,056) of which this application is a continuation-in-part, the cooling properties of these entities on the oropharynx have been described.

(O═)P R₁R₂R₃

The selected compound has a sensory effect on the esophagus that stimulates coolness and counteracts discomfort (heartburn, sour taste, and pain). This sensation is similar to when ice cream is swallowed but lasts longer. A sensation to avoid in a molecule is “cold discomfort.”

The topical formulation for localized delivery of the selected molecule onto nerve endings of the 9^{th} and 10^{th} cranial nerves in the hypopharynx and upper esophagus must have fast onset (≤5 min) and sufficient duration (˜1 hr). The dosage schedule should allow the patient to regain control of the discomfort. Ideally, the active compound is potent, with a unit dose of fewer than 10 mg per administration.

The conventional gelatin-coated pills containing liquid or semi-liquid contents are configured for ease of swallowability. The gelatin coating or shell prevents dispersion of its contents in the oral cavity, avoiding actions on taste buds and a sticky chalky feel in the mouth. The standard gel pill is usually spherical, oblong, oval, elliptical, or almond-shaped and designed to facilitate its passage from mouth to stomach. An accompanying sip of water helps reduce friction and gives volume to the bolus. The gel then glides past the upper esophageal sphincter during the swallow. A fast PTT is desirable for a gel that delivers the active ingredient into the stomach. Typically, the gel is created to minimize pharyngeal residue, as measured by videofluoroscopy.

The inventive step here is to do the opposite, to design a “Shaped-Gel” configuration that will be intercepted, impeded, ensnarled, or trapped in the pharyngeal valleculae and pyriform sinuses before it passes down the esophagus. This design is counterintuitive because the golden rule is to avoid pharyngeal residue. The goal is to prolong the transit time of the Shaped-Gel so the active ingredient has more time to dissolve in saliva, disperse, and reach receptors for cooling. By experiment, the ideal formulation of Shaped-Gel was a torus or a flat rectangle, with a mass of 0.3 to 0.8 g. Flatness was defined as a pill with the shortest axis, preferentially 5 to 45% of the longest axis. A hemi-torus also worked well. In addition, the presence of single or multiple holes in the Shaped-Gel may enhance the available surface area for dissolution. The Shaped-Gel itself was composed of gelatin, glycerol, and water. Gelatin may be replaced by other gelatinous substances and glycerol by other plasticizers (vide infra). The Shaped-Gel does not affect taste buds on the tongue surface. Also, the Shaped-Gel, in contrast to liquid drops, allows the administration of a higher dose of the active ingredient.

The inventive Shaped-Gel should not be confused with pills such as lozenges or pastilles held in the mouth until they dissolve, or troches or tablets that are small, hard, and swallowed whole, or with softgel pills that have a hard or soft shell and also swallowed whole. Instead, the word “Shaped-Gel” will describe an object with a gelatin or gelatin-like coat, shaped deliberately for deposition and retention, with rapid dissolution in the pharynx. The Shaped-Gel dissolves and releases its contents onto the surfaces of the hypopharynx, the upper esophageal sphincter, and the upper esophagus. The effective Shaped-Gel has the correct size and shape to carry the cooling molecule. The Shaped-Gel should be wet for swallowing, and this can be done with saliva or a liquid of 52 mL.

After formulating the Shaped-Gel and swallowing it, the efficacy of the Shaped-Gel is assayed. In principle, the dissolution of the gel can be monitored by videofluoroscopy or by ultrasound, but the gel is not opaque to x-rays or sound. On the other hand, external landmarks on the throat serve as good indicators for locating the cooling. Coolness behind the voice box (i.e., the larynx or behind the thyroid cartilage) indicates that the cooling agent is at the level of the hypopharynx. Cooling at the jugular notch confirms that the cooling agent is below the upper esophageal sphincter. Cooling behind the manubrium indicates that the cooling agent has reached the upper third of the esophagus. If cold discomfort occurs behind the xiphoid process, the cooling agent has penetrated the lower esophagus, and this formulation may not be suitable.

The measurement of drug action should be at the jugular notch. A preferred onset of cooling is ≤5 min, and the preferred duration of action is about 1 hr or more. These parameters are adjustable, for example, by setting the glycerol-gelatin ratio and the amount of water in Shaped-Gel. For example, increasing the gelatin content from 12% to 15% delayed the onset from 2 min to 8 min. The choice of an active ingredient for Shaped-Gel is empirical. An initial criterion is a potency greater than I-menthol at the TRPM8 receptor, as measured by the EC₅₀ on transfected cells. The median effective dose (EC₅₀) measures potency, but the 95% confidence limits of the EC₅₀ have considerable overlap, so potency comparisons have limited value. A second criterion is receptor selectivity. Selectivity means a candidate is active on the TRPM8 receptor but not on TRPV1 or TRPA1. These latter receptors are associated with the perception of pain. A third criterion for choosing a molecule is “good efficacy,” which means that a maximal intensity of the desired pharmacological effect is attainable. Other measurable criteria are the absence of adverse taste and “burning, icy cold” on the throat, features determined by an experiment. For example, although potent, the hexyl and heptyl DIPA-analogs were more likely to produce cold discomfort.

Ideally, the chosen molecule had potency in the range of 0.4 to 1.5% (4 mg/mL to 15 mg/mL) in the Shaped-Gel. Chemically, the molecule should be homogeneous in the gelatin-glycerol-water medium and stable to heat and packaging. If the molecule is water-soluble, then miscibility with the carrier vehicle is improved and facilitates delivery and contact with the target. Using these criteria, the nonyl substitution on DIPA was a preferred embodiment. Other cooling agents in the 1-dialkylphosphoryalkane and p-menthane carboxamides families were screened and selected. As will be appreciated by one of skill in the art, features and preferred embodiments of one aspect of the discovery will also pertain to other aspects of the discovery.

In summary, the proposed design of a Shaped-Gel method of drug delivery relies on a shape that is intercepted in the valleculae and pyriform sinuses above the upper esophageal sphincter. The delay in transit gives the Shaped-Gel cooling ingredient more time to dissolve in saliva and act on the hypopharynx and the esophagus receptors. The cooling agent in Shaped-Gel selects for the TRPM8 receptor. As a result, symptoms of pharyngitis, esophagitis, reflux disease, chest pain, and dyspepsia are relieved.

### BRIEF DESCRIPTION OF THE SEVERAL VIEW OF THE DRAWINGS

FIG. 1. is a diagram of the upper digestive tract and its connections.

FIG. 2. is an anatomic diagram of the upper digestive tract and its connections. Bitter taste is sensed mainly the posterior third of the tongue (marked by a X). The valleculae are at the inferior border of the oropharynx, a the notch formed between the root of the tongue and the epiglottis. The pyriform sinuses are at the level of the larynx, in the hypopharynx. The valleculae and pyriform sinuses collect residue if the ingested item does not go past the upper esophageal sphincer located at the base of the hypopharynx.

FIG. 3. is a drawing of the Shaped-Gel entering the oropharynx. The Shaped-Gel, shaped as a toroid, is designed to be intercepted or trapped in the valleculae. The torus can also be trapped in the pyriform sinus. The trapped Shaped-Gel dissolves in saliva or a small volume of an imbibed liquid.

### DETAILED DESCRIPTION OF THE INVENTION

The present discovery pertains to formulating a cooling agent for localized, topical application to nerve endings. The perception of coolness varies over the body's surfaces. The ocular rim, eye surface, and scrotum are especially sensitive to cold stimuli, followed by the tip of the nose, anogenitalia, jugular notch, flexures of the elbow and knees, and skin about the shoulder blades and ankles. Cold from these areas of the body is localized and discrete. For sites covered with mucous membranes, the oropharynx, nasal cavity, and nasopharynx are more sensitive than the oral cavity. The sensitivity of the hypopharynx and esophagus has not received much attention, but the vigorous response observed here is surprising, unexpected, and startling. If the throat and upper esophagus lumen feel cool, the whole body swiftly feels cool and cold. The coolness in this location is recognized by the brain as core temperature and integrates into mainstream perception. It is like turning on the central air conditioner.

The proposed formulation is useful for treating (e.g., selectively suppressing) sensory discomfort from disorders of the pharyngeal-esophageal tract. The onset of drug effect is rapid 5 min) and of sufficient duration to be therapeutic. There are no other products on the market that match this mechanism of drug action. Consequently, this formulation with its chosen cooling agent may be useful for treating disorders from the esophagus, including pharyngeal discomfort, esophageal discomfort, throat irritation, cough, esophagitis, heartburn, regurgitation discomfort, dysphagia, dyspnea, dyspepsia, chest pain, and acid reflux discomforts.

The formulation comprises a cooling agent, gelatin, glycerol or sorbitol plasticizer, and water. This mixture is warmed to form a liquid solution and then cooled in a mold to form a three-dimensional object. The object's configuration is specific for the proper delivery of the cooling agent to its target receptors on the hypopharynx and esophagus. The object should preferentially be flat, with a short axis that is 5 to 45% of the longest axis. The object should preferentially have one or more holes in its body to increase the surface area for liquids such as saliva or ingested liquid to dissolve the gelatin-glycerol-water formulation. The object should preferentially have a mass of 0.3 to 1.0 g, and more preferentially, 0.4 to 0.6 g. This configuration allows the object to be intercepted, impeded, ensnarled, or otherwise trapped in the pharyngeal valleculae and pyriform sinuses before propulsion past the upper esophagus sphincter. This delay in passage increases the active ingredient's transit time, permits saliva dissolution, and enhances its distribution to target receptors in the hypopharynx and esophagus. A large mass, e.g., >1.5 g, was difficult to swallow. Also, a large mass in the head of the bolus exerts pressure on the opening of the upper esophageal sphincter, causing a faster exit into the lower esophagus. Experiments determined the ideal shape and mass.

The prior art design for gelatin drug delivery systems enhances the swallowability and rapid transit of medicinal objects, not delaying their passage in the throat. The gelatin-glycerol-water object above is called a “Shaped-Gel” to distinguish it from softgels designed for quick transit. For some subjects, a sip of liquid of a small volume, e.g., ≤5 mL, a teaspoonful, may aid the wetting of the Shaped-Gela and facilitates swallowing.

The site of Shaped-Gel effects in the throat of volunteers was identified by asking them the location of cooling, namely, behind Adam's apple, at the jugular notch, or behind the breastbone. These external anatomic landmarks correspond to the hypopharynx and trachea, below the upper esophageal sphincter, and the upper esophagus. Cooling below the body of the breastbone, down to the xiphoid process, was not desirable because of the risk of cold discomfort. If significant cooling was at Adam's apple and the jugular notch, the Shaped-Gel formulation was considered successful for further study and development.

In experiments, the ideal properties for treating the pharyngeal-esophageal surfaces selected were:
The ideal Shaped-Gel formulation weighs 0.3 to 1 g. Smaller sizes get swallowed too quickly, and larger sizes are too difficult to swallow.
The shape of the Shaped-Gel unit is preferably flat, with a short axis that is 10 to 45% of the longest axis. The flat shape favors swallowing and trapping in the recesses of the pharynx. Examples of ideal shapes are toroids and rectangular prisms.
The effective concentration of the cooling agent in the Shaped-Gel is in the range of 3 to 15 mg/g. By effective is meant a cooling agent that has onset in ≤5 min and acts for min with a cooling action that is comfortable and not too strong (to evoke icy cold).
When tested in volunteers with esophageal tract discomfort, several optimized Shaped-Gels alleviated the discomfort of a full meal.
No current medications for disorders of the esophageal tract, such as reflux or indigestion, have this mechanism of action and fast onset of relief.

### Abbreviations and Terminology

Adam's apple. Also known as the laryngeal prominence, it is the lump or protrusion in the human neck formed by the angle of the thyroid cartilage surrounding the larynx and is more prominent in males than in females. Just below the thyroid cartilage is the cricoid cartilage.

Cognitive Fields for Coolness. The perception of coolness varies over the body's surfaces. The ocular rim, eye surface, and scrotum are especially sensitive to cold stimuli, followed by the tip of the nose, anogenitalia, neck, flexures of the elbow and knees, and skin about the shoulder blades and ankles. Cold from these keratinized areas of the body is localized and discrete. For sites covered with mucous membranes, the oropharynx, nasal cavity, and nasopharynx are more sensitive than the oral cavity. The sensitivity of the hypopharynx and esophagus has not received much attention, but the vigorous response observed here is surprising, unexpected, and startling. If the throat and upper esophagus lumen feel cool, the whole body swiftly feels cool and cold. Apparently, the coolness in this location is recognized by the brain as core temperature and integrates into mainstream perception. It is like turning on the central air conditioner.

Cold Discomfort This term describes three types of sensations—“icy cold,” coldness in the chest, and systemic coldness. Icy cold can be felt in the throat and esophagus and can be acutely painful. Coldness in the chest is felt behind the sternum and is equally uncomfortable. Systemic coldness is equivalent to chills and is felt first around the eyes, then the skin of the shoulder blades and ankle. Cold discomfort limits the selection of the active ingredient for localized action on the pharynx and upper esophagus. Therefore, the ideal agent selected must have a circumscribed site of action, and the intensity of the sensation should not cause “icy cold,” coldness in the chest, or systemic chills. An ideal agent is DIPA-1-9 because it is water-soluble, and its pharmacokinetic properties permit access to receptors at the basal layer of the stratified pharyngeal epithelia. In addition, DIPA-1-9 does not enter the systemic circulation or over-activate the cold receptors in the thinner epithelial layers of the larynx and esophagus. Thus, DIPA-1-9 will produce refreshing cool but not cold discomfort.

DIPA compounds DIPA is the abbreviation for 1-[Diisopropyl-phosphinoyl]-alkane]. A number may describe the third alkyl group in the molecule: hence, 4, 5, 6, 7, 8, 9, and 10 correspond to the butyl, pentyl, hexyl, heptyl, octyl, nonyl, and decyl side chain, respectively. These alkanes are linear or “normal [n]” in configuration, with the phosphinoyl group attached to the primary, or “1-” position, of the carbon chain in the third sidechain. An alternative name for these compounds is trialkyl phosphine oxides or 1-dialkylphosphorylalkanes. The compounds' names derive from phosphinic acid or phosphoric acid. Hence the terms “phosphinoyl” or “phosphoryl”, but are equivalent and interchangeable in the context of the DIPA compounds.

Eosinophilic Esophagitis. Eosinophils are a type of white blood cell associated with the regulation of immune functions. Typically, the esophageal mucosa is devoid of eosinophils. But when 15 eosinophils per high-power microscopic field are present, the histologic diagnosis of eosinophilic esophagitis (EE) is established. The signs and symptoms of EE overlap with GERD, heartburn and dysphagia being present, but PPIs are not effective for EE. EE patients receive topical steroids, but consistent drug delivery is problematic [Furuta G T, Katzka D A. Eosinophilic Esophagitis Definition and Differential Diagnosis. N Engl J Med. 2016; 373(17):1640-8]. A topical cooling agent has good potential for relieving the symptoms of EE.

Epigastric Region. The epigastric (above the stomach) region is the abdominal region that is central in location, above the umbilical region, and between the two hypochondriac regions. It is the triangular region below the xiphoid process. Reflux disease produces heartburn, a burning sensation originating from the epigastrium and radiating upwards to the throat and neck.

Epiglottic Vallecula Vallecula is a term for furrow, valley, or depression. The epiglottic valleculae are anatomic depressions located at the base of the tongue next to the median glossoepiglottic fold. The mean vallecular area estimated by barium imaging of humans was 84 mm² and the mean vallecular volume was 1.55 mL (Kim et al. Dysphagia February, 2021. DOI: 10.1007/s00455-020-10106-1). The valleculae can retain saliva and food particles. The vallecula is a landmark for placing the tip of a curved laryngoscope for endotracheal intubation.

Esophagus. The esophagus is a muscular tube about eight to ten inches (25 cm) long that connects the hypopharynx to the stomach. The esophagus runs behind the windpipe (trachea) and in front of the spine. It extends from the cricopharyngeus muscle at the cricoid cartilage level to the stomach's entrance at the level of the diaphragm. Suppose one takes a cross-section of the body in the rostral-caudal direction (vertical axis) at the neck level. The center of the section would be the esophagus. The width would be about 1.5 to 2 cm which is about 10% of the diameter of the trachea. The esophagus is hard to discern among the blood vessels in a cross-section of the thorax, but it is in the center.

Jugular Notch. Also known as the suprasternal notch is the visible external dip in the center of the neck in humans, between the collar bones (clavicles) and above the manubrium (hilt) of the sternum. The trachea lies behind it.

Liquid Volume to Accompany Swallowing a Shaped-Gel. In clinical trials, 240 mL of water is often given to subjects to swallow pills. Smaller volumes, e.g., 100 mL, have been used. In a study of 428 participants, volunteers ingested 40 mL on average [Bar-shalom D et al. Swallowability. Tablets Capsules. 2016; (January):1-4]. In the practice of this invention, subjects receive instructions to sip or use a teaspoon (5 mL) volume for swallowing, if necessary. A swallowed volume of ≥10 mL will promote the rapid opening of the cricopharyngeus muscle [Cook I J et al. opening mechanisms of the human upper esophageal sphincter. Am J Physiol—Gastrointest Liver Physiol. 1989: 257, 748-759] and reduce PTT. This rapid opening reduces the contact time of the active ingredient with the TRPM8 receptors and is not desirable.

Lower Esophageal Sphincter. The lower esophageal sphincter is a specialized segment of the circular muscle layer at the esophageal-gastric junction. Its contraction reduces backflow, and its relaxation increases the backflow of gastric contents into the esophagus.

Menthol lozenges (also called pastilles) have been around since the 1930s and are sold by Halls (Mondelez Global, Canada) at Walmart stores for about $2 per bag of 30. The lozenges are known as “hard candy,” each weighing about 3 g. It is mechanically impossible to cough when the lozenge is in the mouth. The saliva dissolves the lozenge, and there is a cooling effect in the oral cavity and throat because these lozenges contain menthol at 2.5 to 16 mg. The limitations of the lozenge are the harsh taste of menthol and the need to hold the lozenge in the mouth till it completely dissolves (˜30 min). For the higher doses of menthol, there is cold discomfort in the chest behind the sternum. This unpleasant coldness behind the sternum is frightening to some subjects because chills remind people of death. Most likely, it is the menthol dissolved in the saliva that is acting on the esophageal lining. Menthol is rapidly absorbed. Also, the fast pharyngeal transit time (PTT) of 51 sec prevents retention of the mentholated saliva on the upper airway surface. So as soon as the lozenge has completely dissolved, any salutary effects of menthol on the throat also dissipate. There is no evidence that menthol lozenges are used for therapy by patients with esophageal disorders.

Pharyngeal Residues. In up to 20% of elderly patients, pharyngeal particle residue is seen in the valleculae and the pyriform sinuses after swallowing (Eisenhuber et al. Videofluoroscopic assessment of patients with dysphagia: Pharyngeal retention is a predictive factor for aspiration. Am J Roentgenol. 2002; 178(2):393-8). Retention of ingested solids is seen using barium contrast media and videofluoroscopy. The pyriform sinus is a favored site for the deposition of particles.

Pharyngeal Transit Time (PTT) is the time between the arrival of the bolus tail at fauces and the complete passage of the bolus tail through the upper esophageal sphincter. The average time is 1 sec and measured with videofluoroscopy (for example [for example, see Regueiro et al. Influence of Body Height on Oral and Pharyngeal Transit Time of a Liquid Bolus in Healthy Volunteers. Gastroenterol Res. 2018; 11(6):411-5.]

Pyriform Recess. On either side of the laryngeal orifice in humans is a recess termed the pyriform recess (also, piriform sinus, or smuggler's fossa). The pyriform sinuses situate in the hypopharynx. The term “pyriform” means “pear-shaped.” The term smuggler's fossa comes from its use to smuggle small items. Food particles and irregular shapes, such as fishbones, can deposit and be “trapped” in the valleculae and pyriform recess.

Receptive field of a sensory neuron is the region in space in which a stimulus will modify the neuron's firing. The space of the receptive field is in the distribution of the nerve endings. For the epithelium, the nerve endings interdigitate with the cell layers at the basal layer of the epithelium. A receptive field, even though minuscule in area, e.g., about an mm², when activated by the appropriate stimulus, e.g., nociceptive or pruritic, can dominate the attention of the brain and mind. Witness what happens when a sharp pin or sting comes into contact with skin or when a dog is preoccupied with a flea bite.

Softgel. A softgel is an oral dosage form of medication (e.g., see http://dictionnaire.sensagent.lepansien.fr/Capsule (pharmacy)/en-en/.) The capsule consists of a gelatin-based shell surrounding a liquid or solid fill. In recent developments, the gelatin may is replaceable by starch or carrageenan. Softgel capsules combine gelatin, water, an active ingredient, and a plasticizer such as glycerin or sorbitol.

Softgel Shape and Size. Softgel capsules or gel-coated tablets have to quickly pass from the mouth to the stomach. The capsules deliver food supplements (e.g., glucosamine and chondroitin), herbals, vitamins, and minerals because these tablets tend to be large, >0.6 to 1.2 g, and consumers sometimes find them difficult to swallow. Medicinal tablets generally range from 25 to 325 mg per tablet and are easy to swallow with water. Typically, softgels are round, oval, elliptical, or oblong. With a plastic mold, any shape can be chosen and fabricated. Subjects find almond or oval-shaped tablets easier to swallow than round tablets. Tablets with angles, such as diamond, rhomboid, or pentagram shapes, are seldom used because they look difficult to swallow. An oral tablet can range from 25 mg up to 1250 mg.

For the practice of this invention, the Shaped-Gel has a preferred flat shape to be trapped in the valleculae or pyriform sinuses. The three-dimensional shape can be circular, rectangular, oval, or elliptical and have holes or cavities. The weight can range from 0.3 to 1 g, but preferably 0.5 to 0.8 g. The surface area of the preferred embodiment, calculated by mensuration, is expected to be at least 133% greater than the surface area of a sphere of equal volume. The preferred “flat” shape definition specifies that one axis is 5 to 45% of the longest axis and preferably 15 to 25%. A “hole” or multiple holes in the Shaped-Gel facilitates the dissolution of the contents in saliva.

Swallowing (deglutition) is a complex coordinated function wherein food and liquid move from the oral region to the stomach. The individual sensors and effectors for swallowing are well-described in a review by Miller. Developmental Disabilities

Research Reviews: 14: 77-86 (2008).and by Matsuo et al. [Anatomy and Physiology of Feeding and Swallowing: Normal and Abnormal. Phys Med Rehabil Clin N Am. 2008:19(4):691-707] and the Matuso et al. paper is incorporated herein by reference.

Teaspoon. A unit of measure used in cookery. According to the US Code of Federal Regulations § 101.9, a teaspoon is equal to 5 milliliters. This liquid volume may vary slightly among countries like the USA, Australia, and the United Kingdom.

Torus and Toroid. In geometry, a torus (plural tori, colloquially donut) is a surface of revolution generated by revolving a circle in three-dimensional space about an axis coplanar with the circle. If the revolved figure is a circle, the object is called a torus. If the revolved figure is not a circle, it is called a toroid. For this invention, an ideal shape of a Shaped-Gel is a toroid or a hemitoroid.

TRP channels The transient receptor potential (TRP) family of cation channels are peripheral detectors of nociceptive, thermal, and painful stimuli. Many of these receptors are located on the nerve membranes of sensory neurons and respond to chemical irritants and changes in local temperature by activating nerve action potentials. The brain perceives and acts upon these signals. Thus, TRP receptors transduce sensory information, and this transduction system regulates and protects the organism from external irritants.

Upper Esophageal Sphincter. The cricopharyngeus muscle is part of the upper esophageal sphincter mechanism. It is at the junction of the hypopharynx and cervical esophagus, at about the level of C5-C6. At rest, the cricopharyngeus muscle is contracted, like a clenched fist, such that no air enters the esophagus. Upon the initiation of swallowing, the cricopharyngeus muscle relaxes and allows the bolus to pass.

Vallecula. The epiglottic valleculae are two depressions at the base of the tongue, at the inferior border of the oropharynx, and located behind the epiglottis. The valleculae can collect saliva and particles of food.

Videofluroscopy (VFS). VFS is a technique for dynamic radiographic assessment of swallowing function wherein the patient swallows a radiopaque contrast medium such as barium sulfate and the movement of the barium image is tracked by x-rays. Withdrawal from mentholated cigarettes. In tobacco products the addition of menthol reduces the irritation of the smoke and the cooling sensations are refreshing and makes smoking more addictive. Menthol may be banned from cigarettes in the future. For smokers who miss the cooling sensations, a Shaped-gel containing a cooling agent may be a medication for dealing the withdrawal symptoms of abstinence from mentholated cigarettes.

### DIPA Compounds

The discovery relates to a particular compound within the series of compounds known as phosphine oxides (which have the following general formula), and more particularly, an example of the group known as di-alkyl-phosphinoyl-alkanes (herein referred to as “DAPA compounds”) (wherein each of R₁, R₂, and R₃ is an alkyl group).

(O═)P R₁R₂R₃

And more specifically, to one particular 1-diisopropyl-phosphinoyl-alkane (DIPA), 1-Diisopropyl-phosphinoyl-nonane, referred to herein as “DIPA-1-9”.

TABLE 1
Chemical structure of DIPA-1-9

| | Chemical | Formula/ | |
|---|---|---|---|
| Code | Name | Weight | Chemical Structure |
| DIPA- 1-9 | 1-Diispropyl- phosphinoyl- nonane | C₁₅H₃₃OP 260.40 | 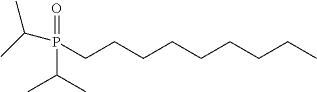 [SMILES: CCCCCCCCCP(=O)(C(C)C)C(C)C] |

DIPA-1-9 is a liquid at room temperature, with a density of ˜0.92 g/cm³ and a boiling point of 112-120° C. Note that DIPA-1-9 is achiral and does not have enantiomers.

By comparison to related DAPA compounds, the Inventor has identified DIPA-1-9 as an exceptional agent for the treatment of sensory discomfort arising from the membranes of the upper esophageal tract and surfaces. The applicant has reported on the efficacy of DIPA-1-9 for the membranes of the nasal cavity, for the transitional epithelium of the ocular surface (U.S. Pat. Nos. 9,642,868 and 9,895,382) and for the oropharyngeal surface. This is the first detailed report of the activities of DIPA-1-9 on the esophagus.

As described herein, DAPA compounds evoke cooling in the throat. This sensation of cool/cold is the desired sensory effect for relieving esophageal discomfort. By topical administration of DAPA, the sensation is localized. The receptive element on neuronal membranes for DIPA-1-9 was characterized as TRPM8, an ion channel receptor. The optimized analogs did not produce stinging, or “icy cold” pain, even when the dose was increased to 12 mg per unit. By choosing a Shaped-Gel delivery method, the activity of the DAPA compound was confined to the throat and upper esophagus, and there was no systemic cooling.

### Chemical Synthesis

DAPA compounds were prepared by the following general method: 100 mL (23.7 g, ˜200 mmol) of sec-butylmagnesium chloride or bromide (isopropylmagnesium chloride or bromide) (obtained from Acros, as a 25% solution in tetrahydrofuran (THF)) was placed under nitrogen in a 500 mL flask (with a stir bar). Diethylphosphite solution in THF (from Aldrich, D99234; 8.25 g, 60.6 mmol in 50 mL) was added drop-wise. After approximately 30 min, the reaction mixture warmed up to boiling. The reaction mixture was stirred for an extra 30 min, followed by a drop-wise addition of the appropriate n-alkyl iodide solution in THF (from TCI; 60 mmol in 20 mL). In the case of DIPA-1-9, the n-alkyl halide was 1-iodononane. The reactive mixture was then stirred overnight at room temperature. The reaction mixture was diluted with water, transferred to a separatory funnel, acidified with acetic acid (˜10 mL), and extracted twice with ether. The ether layer was washed with water and evaporated (RotaVap Buchi, bath temperature 40° C.). The light brown oil was distilled under high vacuum. The final products, verified by mass as determined by mass spectrometry, were clear liquids that were colourless or slightly pale yellow. The compounds prepared by these methods are shown in Table 2.

TABLE 2
Chemicals prepared and tested.

| Code | Chemical Name | Chemical Structure |
|---|---|---|
| DIPA-1-5 | 1-Di(isopropyl)- phosphinoyl-pentane | 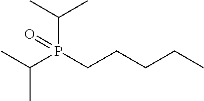 |
| DIPA-1-6 | 1-Di(isopropyl)- phosphinoyl-hexane | 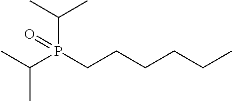 |
| DIPA-1-7 | 1-Di(isopropyl)- phosphinoyl-heptane | 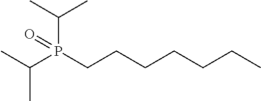 |
| DIPA-1-8 | 1-Di(isopropyl)- phosphinoyl-octane | 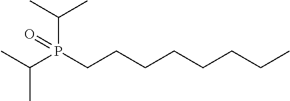 [SMILES: CCCCCCCCP(=O)(C(C)C)C(C)C] |
| DIPA-1-9 | 1-Di(isopropyl)- phosphinoyl-nonane | 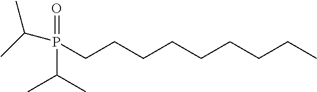 [SMILES: CCCCCCCCCP(=O)(C(C)C)C(C)C] |
| DAPA-2-4 | 1-Di(sec-butyl)- phosphinoyl-butane | 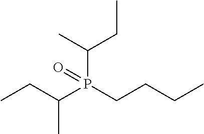 |
| DAPA-2-6 | 1-Di(sec-butyl)- phosphinoyl-hexane | 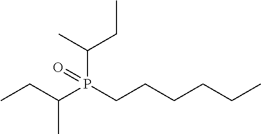 [SMILES: CCCCCCP(=O)(C(C)CC)C(C)CC] |
| DAPA-2-7 | 1-Di(sec-butyl)- phosphinoyl-heptane | 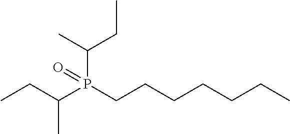 [SMILES: CCCCCCCP(=O)(C(C)CC)C(C)CC] |
| DAPA-2-8 | 1-Di(sec-butyl)- phosphinoyl-octane | 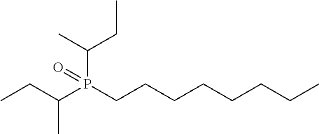 [SMILES: CCCCCCCCP(=O)(C(C)CC)C(C)CC] |
| 3,4-6 | 1-(Isopropyl-sec- butyl)-phosphinoyl- hexane | 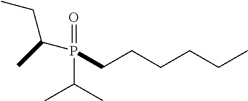 |
| 3,4-7 | 1-(Isopropyl-sec- butyl)- phosphinoyl-heptane | 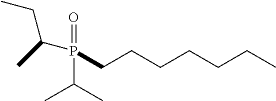 |
| DAPA-3-1 | 1-di(iso-butyl) phosphinoyl-pentane | 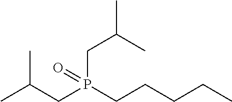 |
| DAPA-3-2 | 1-Di(sec-butyl)- phosphinoyl- 3-methyl-butane | 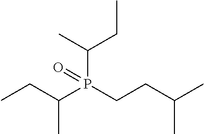 |

Compositions The 3,4-X series are “mixed” isopropyl-sec-butyl compounds (see below). These were synthesized by Dr. Jae Kyun Lim of Dong Wha Pharmaceuticals, using the method described below.

Briefly, as illustrated in the following scheme, triethyl phosphite (A) was reacted with sec-butyl magnesium bromide (B) and then hydrolysed with dilute hydrochloric acid to give the mono-alkyl compound (C). The product (C) was then reacted isopropyl magnesium bromide (D) to give the di-alkyl compound (E), which was then reacted with a suitable alkyl iodide (F) to give the target trialkyl phosphine (G).

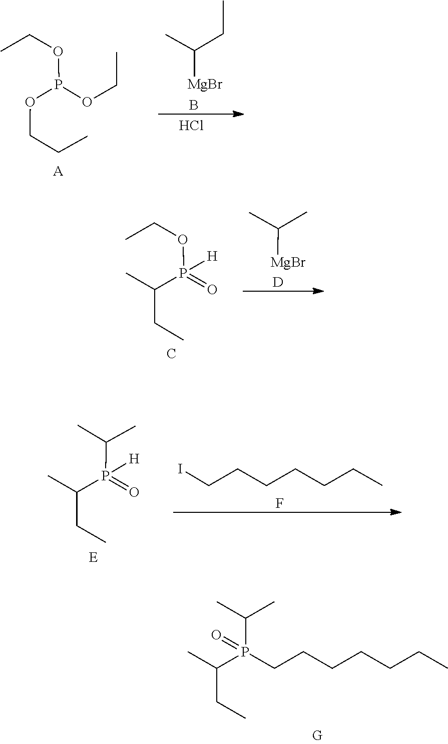

The DIPA compounds are colorless liquids with a density less than water. These structures differ from those described by Rowsell and Spring U.S. Pat. No. 4,070,496 because '496 structures have their “head” (phosphine oxide group) covered by larger, more lipophilic groups. The applicant noted that '496 did not include the di-isopropyl analogs. The applicant synthesized these analogs (which are achiral, by contrast to the structures of '496 which are >95% chiral). The applicant found that, by minimizing the two alkyl side chains to di-isopropyl, the “head” of the prototypical molecule now is more polar (hydrophilic) and more miscible in the polar environment of water. This increased water-solubility is striking (Table 3).

TABLE 3
Water solubility (mg/ml) of 1-dialkylphosphinoylalkanes
(R₁R₂R₃P═O).

| No. | | | | | | | | |
|---|---|---|---|---|---|---|---|---|
| Carbons | 13 | | 14 | | 15 | | 16 | |
| R₁, R₂ | R₃ | | R₃ | | R₃ | | R₃ | |
| di-sec- | pentane | 22 | hexane | 8 | heptane | <3 | octane | <3 |
| butyl- | | | | | | | | |
| isopropyl- | hexane | 25 | heptane | 20 | octane | <3 | nonane | <3 |
| sec-butyl- | | | | | | | | |
| di- | heptane | >300 | octane | >300 | nonane | >300 | decane | <3 |
| isopropyl- | | | | | | | | |

In one embodiment, the composition comprises DIPA-1-9 at a concentration of 0.4 to 1.5% wt/wt. In one embodiment, the composition is a Shaped-Gel composition, and comprises DIPA-1-9 at a concentration of 5 to 15 mg/mL The composition may be provided with suitable packaging and/or in a suitable container. For example, the composition may be in the form of unit oral dosage unit, for example, in a blister pack.

A preferred delivered volume is 0.4 to 0.6 g of a gel. For a gel a preferred concentration of the cooling compound is 5 to 15 mg/g. A preferred amount of the compound delivered at the site of the application is 1 to 10 mg.

One aspect of the present discovery pertains to DIPA-1-9 for use in a method of treatment (e.g., targeted treatment) of certain disorders (e.g., a diseases), as described herein. In one embodiment, the medicament comprises DIPA-1-9. In one embodiment, the medicament comprises DIPA-1-9 formulated as a Shaped-Gel. Another aspect of the present discovery comprises administering to a patient in need of treatment a therapeutically effective amount of DIPA-1-9, preferably in the form of a pharmaceutical composition. In one embodiment (e.g., of use in methods of therapy, of use in the manufacture of medicaments, of methods of treatment), the treatment is the treatment of esophageal discomfort caused by reflux or inflammation. The term as used herein, relates to unpleasant sensations of heartburn, epigastric pain, regurgitation, hoarseness, cough, dysphagia, bloat and belching.

### Treatment Objectives for Pharynx-Esophagus

The term “treatment,” as used herein in the context of treating a disorder, pertains generally to treatment of a human or an animal (e.g., in veterinary applications), in which some desired therapeutic effect is achieved, for example, the inhibition of the progress of the disorder, and includes a reduction in the rate of progress, a halt in the rate of progress, alleviation of symptoms of the disorder, amelioration of the disorder, and cure of the disorder. Inclusive of such treatments are reduction of sensitivity, of hypersensitization, and desensitization phenomena. Treatment as a prophylactic measure (i.e., prophylaxis) is also included. For example, use with patients who have not yet developed the disorder, but who are at risk of developing the disorder, is encompassed by the term “treatment.”

The term “selective” in pharmacological terminology pertains to a molecule that, among a group of structurally related congeners, exhibits unusual qualitative properties that distinguishes it from the other analogs. For example, DIPA1-9 does not have a strong metallic taste, but this taste is present in DIPA-1-7 and DIPA-1-8 and other analogs. Thus, DIPA-1-9 is more selective in its pharmacological actions.

Another aspect of the selective properties of DIPA-1-9 is the low degree of “cold discomfort” compared to the related analogs. DIPA-1-9 can act on surfaces without problems of stinging, irritancy, and pain in the throat or excessive cold behind the sternum.

In one embodiment (e.g., of use in methods of therapy, of use in the manufacture of medicaments, of methods of treatment), the treatment is treatment (e.g., selective treatment) of: esophageal tract discomfort; esophageal discomfort; throat irritation; cough; heartburn; chest pain; discomfort of regurgitation or a sour, acrid taste in the throat; or inflammation and pain of esophageal tissues. In an another aspect treatment, the DIPA-1-9 is used to stimulate coolness receptors and produce signals that will prevent dysphagia, sense of bloat, belching and hiccups.

In one of the embodiments, the target, tissue for DIPA-1-9 is located on an esophageal surface and the sensory discomfort located on an esophageal surface is caused by reflux of stomach contents (e.g., gastroesophageal reflux) or by esophagitis. In one embodiment, the esophageal tract discomfort is caused by inflammatory exudates in the airways or the pharynx (e.g., associated with asthma, an obstructive pulmonary disorder, etc.). In one embodiment, the esophageal tract discomfort is associated with belching, a sense of bloat, or globus. In one embodiment, the treatment is treatment of esophageal discomfort. And laryngopharyngeal reflux. In one embodiment, the esophageal discomfort is associated with reflux of stomach contents. In one embodiment, the esophageal discomfort is associated with gastroesophageal reflux. In one embodiment, the treatment is of throat irritation. In one embodiment, the treatment is treatment of cough or the urge to cough. In one embodiment, the treatment is treatment of heartburn. In one embodiment, the treatment is treatment of chest pain.

DIPA-1-9 in Shaped-Gel may be used as a diagnostic agent for the differential diagnosis of chest pain. Currently, a simple diagnostic tool is not known. A DIPA-1-9 Shaped-Gel can be administered orally, e.g., swallowed with a sip of water. If the pain is of esophageal origin, the chest pain should be relieved. But, if the pain is cardiac pain, then the DIPA-1-9 Shaped-Gel will not be effective.

Routes of Administration and Dosing

The pharmaceutical composition comprising DAPA compounds such as DIPA-1-9 may suitably be administered to a subject topically, for example, as described herein. The term “topical application”, as used herein, refers to delivery onto the surface of the tongue, then to the lumenal surfaces of the pharyngx and esophagus.

The preferred formulation a DAPA is as a Shaped-Gel. Other ingredients that may be included are preservatives, lubricants, stabilzers, masking agents, coloring agents, and flavoring agents. The formulation may further comprise other active pharmacological agents. If formulated as discrete units (e.g., vials, pre-wrapped units), each unit contains a predetermined amount (dosage) of the compound.

Suitable carriers, diluents, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 5th edition, 2005. The formulations may be prepared by any methods well known in the art of pharmacy. Such methods include the step of bringing into association the compound with a carrier which constitutes one or more accessory ingredients. In general, the formulations are prepared by uniformly and intimately bringing into association the compound with carriers (e.g., liquid carriers).

The gelling agent employed includes but is not limited to gelatin, agar, algin, carrageenan, guar gum, gum arabic, locust bean gum, pectin, and modified starch, and mixtures thereof. In one of the preferred embodiments of the present disclosure the gelling agent used is gelatin.

The plasticizer is selected from the group consisting of glycerol, sorbitol and mixtures thereof. In one of the preferred embodiments of the present disclosure the plasticizer is glycerol. The plasticizer is mixed with the gelling agent and a smaller amount of water.

It will be appreciated by one of skill in the art that appropriate dosages of DAPA and compositions comprising DIPA-1-9, can vary from patient to patient. Determining the optimal dosage will generally involve the balancing of the level of therapeutic benefit against any risk or deleterious side effects. The selected dosage level will depend on a variety of factors including, but not limited to, the activity of the DAPA, the route of administration, the time of administration, the duration of the treatment, other drugs, compounds, and/or materials used in combination, the severity of the disorder, and the species, sex, age, weight, condition, general health, and prior medical history of the patient. The amount of DAPA and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, preferably on an “as-need” or pro re nata basis throughout the course of treatment. Methods of determining the most effective means and dosage of administration are well known to those of skill in the art and will vary with the formulation used for therapy, the purpose of the therapy, the target receptors being treated, and the subject being treated. Single or multiple administrations can be carried out with the dose level and pattern being selected by the patient, treating physician, veterinarian, or clinician.

In the present, preferred embodiments, the dosage of the DAPA can range from 1 mg to 15 mg per dose, with a preferred dosage of 5 to 10 mg per Shaped-Gel. This is equivalent to a concentration of 5 to 15 mg/g of the active ingredient in the Shaped-Gel matrix.

### Pharynx-Esophagus and its Disorders

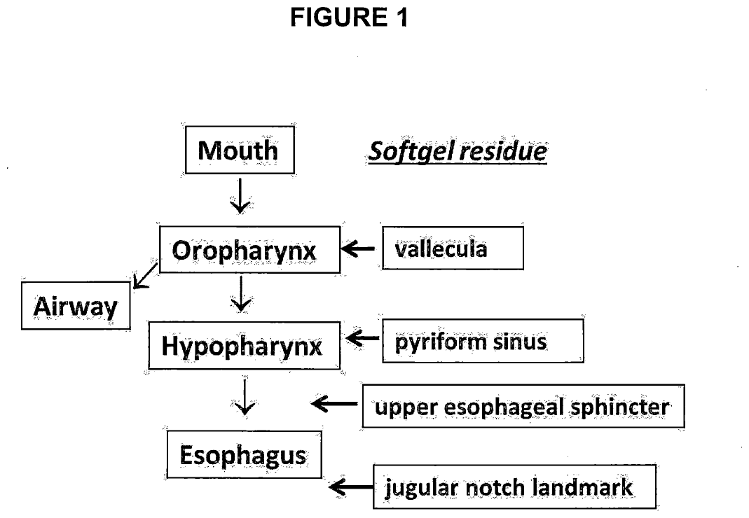

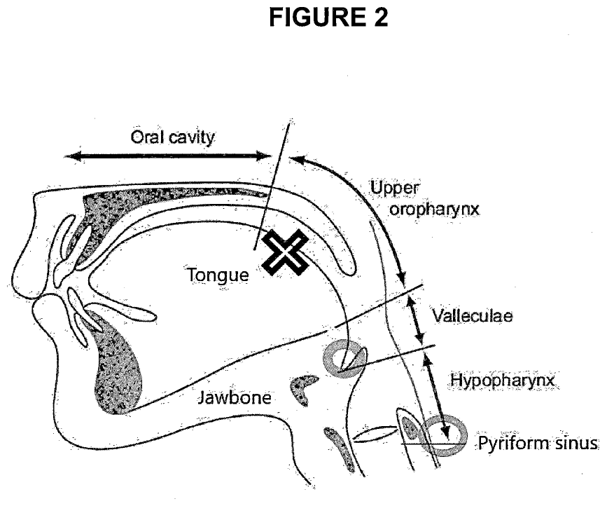

Air enters the nasal cavity and goes to the trachea. Food and liquids enter the mouth and go past the pharynx and into the esophagus. The esophagus connects the upper and lower esophageal sphincter. In this application, the emphasis is on targeting the hypopharynx and upper third of the esophagus with DIPA Shaped-Gels (FIG. 1). The pharynx is divided into naso-, oro-, and hypo-, with the relative positions shown in FIG. 2. The oropharynx reaches from the soft palate to the level of the hyoid bone. The hypopharynx (also called the laryngopharynx) is between the hyoid bone and the cricoid cartilage. Stratified epithelia line the pharyngeal and esophageal surfaces. By contrast, a single layer of pseudostratified epithelium lines the respiratory epithelia of nasopharynx, larynx and trachea.

An astounding traffic load passes through the lumen of the oropharynx. On an average day, a young adult breathes 12,000 L of air, drinks 2 L of fluids, secretes 1 L of saliva, and eats 2 kg of food. These activities are constant, with about 15 breaths and one swallowing movement per min during the waking hours. For survival, the organism must coordinate traffic flow so that food and liquids go down the esophagus and the air gets directed into the airways.

The oral cavity contains specialized structures for mastication and taste, such as teeth, gums, and tongue. The salivary glands provide saliva to lubricate and help propel the food bolus into the pharynx. Swallowing a bolus is a complicated muscular reflex activity that requires six cranial nerves and twenty-five muscle groups to cooperate. Thermosensation is not a high-ranking protective reflex in the mouth which tolerates liquids hot enough to cause pain on the skin. Cooling liquids, by contrast, are important in the regulation of thirst and a source of positive reinforcement when enjoying ice cream and popsicles. All these events occur in the oropharynx [see Eccles et al. Cold pleasure. Why we like ice drinks, ice lollies, and ice cream. Appetite, 71, 357-60, 2013]. Sensory nerves closely monitor temperatures at the oral cavity-pharynx junction. When the external ambient temperature is high or after strenuous exercise, drinking a cooling liquid is instantly pleasurable and relieves thirst, dryness, and discomfort.

The pharynx has strong constrictor muscles, arranged as a vice and designed to grab masticated contents and push the bolus into the esophagus. The anatomy is complicated. This system has two essential valves: the epiglottis, which closes during swallowing, and the upper oesophageal sphincter (UES), which opens to allow the contents to enter the esophagus, then shuts to prevent reflux. Pharyngeal contractions usually flush and empty their debris load and create negative pressure that helps suck contents from the nasal cavity and nasopharynx. Well-toned pharyngeal muscles are essential for maintaining the patency of the airways, allowing smooth airflow. Dysfunction with age and brain impairment will cause dysphagia, an increased risk of pneumonia, cough, dyspnea, snoring, and sleep apnea.

The narrowest point of the pharynx, shown, for example, in the magnetic resonance imaging studies of Daniel et al. [“Pharyngeal dimensions in men and women,” Clinics (Sao Paulo) 62, 5-10, 2007] has a cross-section of about 1 cm². The pharyngeal surface at the tongue base and the posterior wall is about 3 to 5 cm². This area is the desired target for drug delivery for the methods described herein. The goals are to lodge the Shaped-Gel in this space, in the valleculae and pyriform sinuses, delay the transit of the Shaped-Gel, allow the Shaped-Gel to dissolve in saliva, and distribute the cooling agent onto the mucosa and access the TRPM8 receptors.

After receiving afferent signals from specific neuronal receptive fields, the brain coordinates pharyngeal-esophageal traffic via effectors (Table 4). Food is masticated, lubricated, and the bolus is pushed down the esophagus in the blink of an eye. All this in a millisecond, at about 35 cm/sec. Afferent signals in the mouth and rostral tongue come via the trigeminal nerve (5^{th}) and hypoglossal nerve (8th). Signals from the oropharynx and posterior surface of the tongue arrive from the glossopharyngeal nerve (9^{th}) and signals from the hypopharynx and upper esophagus come from the vagus nerve (10^{th}) and spinal afferents. The brainstem nuclei recognize discrete topography. All these afferent nerves detect thermosensory and nociceptive stimuli.

TABLE 4
Neuronal Circuity of Afferents

| Afferent nerve | Brain Nuclei | Functions | Disorders |
|---|---|---|---|
| 5^{th} and 8^{th} | spinal trigeminal | mastication, | burning mouth syndrome |
| | nuclei | salivation, taste | |
| 9^{th} | para-trigeminal, dorsal nucleus of the | taste, thirst, | dysphagia |
| | vagus | swallowing | |
| 10^{th} | n. ambiguus, n. tractus solitarius | cough | nausea, dysphagia |
| (pharyngeal) | | | |
| 10^{th} | n. ambiguus, n. tractus solitarius | thermosensory | nausea, reflux discomfort, |
| (esophageal) | | | chest pain, cold discomfort |
| spinal nerves | | peristalsis | reflux discomfort, chest |
| | | | pain, cold discomfort |

The surface cells of the pharynx and esophagus have a high turnover and are sensitive to injury. These cells are susceptible, for example, to inspired or ingested pollutants or toxins, to acid and pepsin from gastric juices, or exudates from the lungs. Disorders manifest themselves as globus (the feeling of a lump in the throat), difficulties in swallowing (dysphagia), difficulty in breathing (dyspnea), hoarseness, various forms of pain, heartburn, itch, cough, and redness and swelling of the pharyngeal mucosa. If airflow or digestion is impaired, there is anxiety.

Chest pain, accompanied by palpitations, sweating, shortness of breath, and choking sensations, is a common symptom that provokes a patient to see a physician or seek admission to an Emergency Department. The physician's immediate priority in examining the patient is to find any life-threatening cardiovascular conditions. If chest pain warrants hospital admission, expenses increase because of physician time, diagnostics such as serum enzyme assays, electrocardiograms, and radiotracer studies on heart function. The median cost of hospital admission for a patient with chest pain was US$7340 [Coley et al., Economic burden of not recognizing panic disorder in the emergency department. J. Emergency Medicine 36: 3-7. 2009]. Each year, at least 6.4 million Americans visit the Emergency Department with complaints of chest pain and related symptoms, but few exhibit an underlying cardiovascular etiology; the others have non-cardiac chest pain (NCCP). Chest pain is the second most common reason for an Emergency Department visit, the first reason being stomach and abdominal pain [see, e.g., see Table 8 in Pitts et al., National Hospital Ambulatory Medical Care Survey: 2006 emergency department summary”, National Health Statistics Reports, Vol. 7, pp. 1-38, 2006]. Natsui et al. recently reviewed records of 38,778 patients admitted to the Emergency Department of Kaiser Permanente Health group in California.

There are multiple causes of NCCP, including pectoral muscle strain, pulmonary disorders, indigestion, panic disorders, and, most frequently, esophageal dysfunction such as GERD [Amsterdam et al., Testing of low-risk patients presenting to the emergency department with chest pain: a scientific statement from the American Heart Association. Circulation, 122: 1756-1776, 2010]. The esophagus is located in the center of the thorax, next to the big blood vessels and heart. If there is irritation inside the esophagus, the sensation may feel like cardiac pain. Standard proton pump inhibitor drugs such as esomeprazole have minimal efficacy in suppressing unexplained chest pain, and the onset of drug effect requires at least several days [Flook et al., Acid-suppressive therapy with esomeprazole for relief of unexplained chest pain in primary care: a randomized, double-blind, placebo-controlled trial, Amer. J. Gastroenterol., 108: 56-64, 2013]. A Shaped-Gel may be valuable for diagnosing and treating chest pain and differentiating between heartburn, angina, and acute cardiac dysfunction.

Cold Discomfort. One aspect of the discovery here is that many of the compounds tested evoke coolness and cold of different intensities. One level of intense cold is painful to the throat. The sensations are akin to rapid drinking of cold water equilibrated with ice chips. If the drink has acid added, for example, with lemonade, the cold is accentuated. Penetrating and intense cold on the throat's surface is uncomfortable and aversive. The term “icy cold” describes this adverse event in the throat. To experience icy cold: Take a glass of water equilibrated (after stirring) with ice chips—a temperature of about 4° C. Then, start sipping the water at about one sip per second. The first five sips are pleasant, but by 5 to 10 sips, the throat feels a dull cold, and after about 10 to 15 sips, the icy cold in the throat becomes unpleasant. The icy cold is in the chest, halfway down to the stomach. These unpleasant sensations constitute “cold discomfort.”

Large doses of I-menthol (>16 mg per candy) produce cold behind the sternum, in the center of the thorax. Chills may accompany these sensations. The cooling agent, dissolved in saliva, most likely distributes and activates cold receptors in the esophageal lining. The number of cell layers on the esophageal epithelium is less than that of the pharynx. These sensations of cold, if not expected by the test subject, can alarm and be viewed as unpleasant. The substernal chills, typically considered unpleasant, may be useful in counteracting the discomfort of chest pain.

The two types of “cold discomfort” described here, icy cold and substernal cold, limit the selection of the active ingredient for localized action on the pharynx and esophagus. Therefore, the ideal agent must have a circumscribed site of action, and the intensity of the sensation should not cause an “icy cold” in the throat or coldness in the chest.

When ice cream is in the mouth, there are pleasant cooling and sweet sensations on the tongue and the walls of the mouth. When the ice cream gulps down, there is a brief robust, refreshing sensation on the back of the mouth. This sensation of swallowing ice cream is equivalent to sipping a “milkshake” or “smoothie.” An ideal sensation for a Shaped-Gel is maintaining an ice cream feeling in the throat. This Haagen-Dazs type of ice cream sensation is optimal for treating airway disorders and relieving pharyngeal-esophageal discomfort. This sensation is called an “ideal cool” for reducing throat and chest discomfort.

Why are the sensations of sipping ice cream different from drinking ice water? In both situations, the temperature of the contents in the throat is about the same, yet it is seldom possible to get unpleasantly cold in the throat with ice cream! One explanation is that the thermal conductivity of the oils and fats that make up ice cream differs from water. For example, the thermal conductivity value of olive oil is 0.17 W/m·K, and that of water is 0.58 W/m·K. Ice water, with higher thermal conductivity (and higher thermal mass), abstracts more heat than ice cream. Therefore, the rate of heat abstraction from the throat's surface determines the perception. When it is too rapid or continuous, there is cold discomfort.

On the other hand, a smooth heat abstraction rate produces a refreshing sensation. Experimentally, ice cream with a high cream content, such as Haagen-Dazs vanilla, best elicits ideal cool. The pharmacological goal is to identify a chemical sensory agent (i.e., a compound that does not abstract heat) that produces an ideal cool and not cold discomfort. Surprisingly and unexpectedly, some DIPA, especially DIPA-1-9 at the optimized concentrations of 5 to 10 mg/mL, elicits a perfect cool in the oropharynx and esophagus but without cold discomfort. By contrast, the sensations of DIPA-1-7 are more intense, and higher concentrations, e.g., 5 mg/mL, will cause an icy cold.

Dysphagia (swallowing dysfunction): Older adults, stroke victims, and subjects with Parkinson's disease or head and neck cancer frequently have difficulty swallowing. Dysphagia describes when a bolus does not transfer properly and efficiently from the pharynx to the esophagus. Aspiration pneumonia occurs when particles enter the airways and is a major economic burden when caring for such victims. Sensory stimulants such as black pepper, capsaicin-like mimics (the active ingredients of chili pepper) administered with a nebulizer, and menthol solutions given by a nasal tube, shorten the latency for a swallowing reflex in the elderly and thus may be useful to reduce the risks of aspiration pneumonia [Ebihara et al., Sensory stimulation to improve swallowing reflex and prevent aspiration pneumonia in elderly dysphagic people”, J. Pharmacol. Sci., 115, 99-104, 2011]. A condition related to aspiration pneumonia is aspiration pneumonitis, when the substances entering the airways come from the esophagus and not the oral cavity.

The studies of Ebihara et al. [vide supra] use agents as aerosols or liquids delivered via a nasal tube. The actual sensory event for enhancement of clearance reflexes was not defined. Potent menthol and peppermint oil confectionery, such as Altoids®, are sensory stimulants in the oral and nasal cavities. Menthol lozenges, weighing about 2.7 to 3.4 g each, containing 5, 7, or up to 10 mg of menthol in a sugar-dye matrix, are sometimes used as oral stimulants but have limited efficacy because of their limited efficacy their harsh taste. Wei described certain N-alkyl-carbonyl-amino acid esters for treating throat discomfort and airway irritation [U.S. Pat. Nos. 8,426,463, 8,476,463]. A Shaped-Gel may be helpful for dysphagia as a stimulant for pharyngeal sensitivity.

Nausea. Nausea is a sense of discomfort associated with an urge to vomit. Nausea is a symptom of motion-sickness, some forms of chemotherapy, over-eating and indigestion. The urge to vomit (emesis) comes from the stomach area, but nausea can be present without emesis. Vagal afferents from the gastrointestinal tract provide the signals for nausea, and the brain nuclei for integration of this sensation are in the nucleus ambiguus and nucleus tractus solitarius pathways. A Shaped-Gel by providing cooling signals via 9^{th} and 10^{th} afferents should relieve nausea, especially if the nausea is associated with indigestion and over-eating.

Reflux disease. Gastric juices entering the esophagus towards the airways cause gastroesophageal reflux disease (GERD), laryngopharyngeal reflux disease (LPR), non-erosive reflux disease (NERD), non-cardiac chest pain (NCCP), and functional dyspepsia (FD). These foodway disorders are sub-divided into “organic” and “functional.” In functional GERD and FD, objective signs of esophageal mucosal erosion are seldom seen but account for 70% of heartburn cases. Gastroenterologists and cardiologists see patients with GERD and dyspepsia because of epigastric pain. The predominant symptoms are heartburn, regurgitation, and non-cardiac chest pains [Oustamanolakis et al., Dyspepsia: Organic vs. functional. J. Clin. Gastroenterol., 46, 175-190, 2012]. The sensations of heartburn and non-cardiac chest pain are primarily of esophageal origin and not cardiac dysfunction. Heartburn is a burning feeling in the chest just behind the breastbone that occurs after eating and lasts a few min to several hours. The substernal burning sensations tend to radiate up into the neck, come in waves, and feel more as burning than as pain. Heartburn is sometimes described as chest pain and exaggerated by postures that promote regurgitation, such as bending over or lying on one's back. Heartburn is felt in the midline and not on the lateral sides of the chest. Accompanying sensations include burning at the back of the throat with sour, acidic, or salty-tasting fluids in the mouth and throat; difficulty in swallowing, and feelings of food “sticking” in the middle of the chest or throat. Otolaryngologists see LPR patients because acid and pepsin enter the pharynx, larynx, Eustachian tubes, and nasal sinuses. Reflux diseases may cause chronic cough, sore throat, persistent hoarseness, larynx edema, and repetitive throat clearing. Examination of the larynx may show red and swollen mucosae about the voicebox. Unifying mechanisms for GERD and LPR were elegantly explained by Park et al. [Modified Reflux Scintigraphy Detects Pulmonary Microaspiration in Severe Gastro-Esophageal and Laryngopharyngeal Reflux Disease. Lung 2021; 199:139-45]. Using a sophisticated scintigraphy technique with radioactive technetium, he showed that gastric contents could become aerosols and travel up the esophagus past the upper esophageal sphincter. Common symptoms of GERD and LPR may include heartburn, regurgitation, chest pain and fullness, discomfort, early satiation, bloating, belching, nausea, vomiting, or pain.

Reflux disorders are managed primarily with acid-suppressive drugs, supplemented if necessary with antibiotics to eradicate H. pylori, prokinetic agents, fundus-relaxing drugs, antidepressants, and psychological interventions. The GERD symptoms alone are sufficient criteria for a patient to be put on an 8-week course of proton-pump inhibitors (PPI) without further diagnostic workup. There are no direct methods to treat the irritation of the esophageal nerve endings by acid and pepsin. A Shaped-Gel acting via TRPM8 receptors may alleviate reflux and satiation discomfort.

In the context of the present discovery, the goals were to:
a) Identify and define an active compound with a precise sensory effect on the esophagus that stimulates coolness and counteracts discomfort (heartburn, sour taste, and pain). This sensation will not produce discomfort but instead generate a sensation similar to when ice cream is swallowed but lasting longer. A sensation to avoid is “cold discomfort.”
b) Develop a topical formulation for localized delivery of the active compound onto targets of the nerve endings of the 9^{th} and 10^{th} cranial nerves in the hypopharynx and upper esophagus.
c) Define a drug action with fast onset and sufficient duration (active for at least one hour), with a dosage schedule that can be therapeutically beneficial, thus allowing the patient to regain control of the discomfort. Ideally, the active compound is potent, with a unit dose of less than 10 mg per administration.
d) Use this medication for short-term (acute) and long-term (chronic) conditions to reduce hypersensitivity to irritant stimuli.

These objectives are met with a Shaped-Gel formulation of a cooling agent.

### Mechanism of Action: Targets on Hypopharyngeal and Esophageal Surfaces

The targets for drug delivery are the TRPM8 nerve endings on the lumenal surfaces of the pharynx and esophagus, particularly the hypopharynx, the esophageal sphincter, and the upper third of the esophagus. The neuronal receptive fields of the preferred targets are on the afferents of the 9^{th} [glossopharyngeal], 10^{th} [vagus], and spinal afferents. The area of the target is several cm². For comparison, the oral cavity surfaces are at least 10× larger. Thus, a chewing gum delivery system will not work because the cooling agent is not focused on the delivery site but dispersed onto the buccal cavity of the mouth.

The binding sites of agonists on the TRPM8 receptor were identified by cryo-electron microscopy. Agonists act on allosteric sites to facilitate the opening of ion channels. TRPM8 immunoreactive fibers are present in the pharynx but not on the epiglottis [Sato, T. et al. The distribution of transient receptor potential melastatin-8 in the rat soft palate, epiglottis, and pharynx. Cellular and Molecular Neurobiology, 33:161-5, 2013]. The hypopharynx is the part of the pharynx that reaches from the hyoid bone to the lower border of the cricoid cartilage. The pharynx is a continuous funnel-shaped inverted trapezoid tube [Daniel et al., 2007] with a surface area of about 10 to 15 cm². The scarcity of TRPM8 nerve endings in the lower airway is clearly shown by Hondoh et al. (Brain Res. 1319:60-9, 2010). The neuronal cell bodies of the 10^{th} nerve are in the nodose ganglion (NG). The neuronal cell bodies of the 9^{th} nerve are in the jugular (JG) and petrosal ganglia (PG). Hondoh et al. using an anti-sense method found that TRPM8 cell bodies locate in JG and PG, but not NG. By contrast, TRPA1-containing neurons locate in all three ganglia. For the upper esophagus, the TRPM8 nerves are in vagal and spinal afferents [Yu X et al. TRPM8 function and expression in vagal sensory neurons and afferent nerves innervating guinea pig esophagus. Am J Physiol-Gastrointest Liver Physiol. 2015: 308(6), G489-496]. The esophageal nociceptors [Ru et al. Adenosine-induced activation of esophageal nociceptors. Am J Physiol—Gastrointest Liver Physiol. 2011; 300(3):485-93] are not linked directly to the TRPM8 neuronal systems. In summary, the targets for the Shaped-Gel are the receptive fields of the 9^{th} and 10^{th} cranial nerves and the spinal afferents of the upper esophagus.

The topographical proximity of the two afferent systems, cooling and nociception, is notable. In the esophagus, the cooling and the nociceptive receptors overlap in the upper third of the esophagus, but the afferent information occurs in separate sets of fibers. The coding of the signals is modality-specific. When the signals reach the brain nuclei, the information integrates. The cooling system suppresses nociception. The esophagus is aligned posteriorly (behind) the trachea. Thus, Shaped-Gel cooling of the esophagus at the levels of the cricoid cartilage, jugular notch, and manubrium can suppress cough signals from the trachea.

### Delivery, Onset, Duration of Action

Swallowing occurs in the blink of an eye, as the bolus moves from mouth to esophagus in milliseconds. Pharyngeal Transit Time (PTT) is the time between the arrival of the bolus tail at fauces and the complete passage of the bolus tail through the upper esophageal sphincter. The average time is ≤1 sec and is measured with videofluoroscopy [for example, see Regueiro et al. Influence of Body Height on Oral and Pharyngeal Transit Time of a Liquid Bolus in Healthy Volunteers. Gastroenterol Res. 2018; 11(6):411-5]. The challenge of any invention is to deliver and retain a sensory agent on the pharyngeal target surfaces. The active ingredient cannot be delivered as solid particles, as that would cause irritation and elicit coughing, so delivery of an agent should be in a liquid or indirectly dissolved in saliva.

The idea here is to use a Shaped-Gel to deliver a cooling agent onto the valleculae, pyriform sinuses, upper esophageal sphincter, and the upper third of the esophagus. The Shaped-Gel's ingredient dissolves and has an onset of about five min. Relief of the symptoms lasts for at least one hour. With practice and familiarity, the subject learns to take the formulation on an “as needed” (p.r.n.) basis. The fast onset allows patient control of esophageal discomfort and reduces psychogenic factors (e.g., anxiety) associated with throat and chest discomfort.

### Molecular Target, Specificity, Selectivity of TRPM8 Agonists

There is a general acceptance that the ion channel TRPM8 is the principal physiological element that transduces to the brain the cooling effects of agents such as menthol and icilin [McKemy et al., Identification of a cold receptor reveal a general role for Trp channels in thermosensation, Nature, 416, 52-58, 2002]. TRPM8 is a protein with 1104-amino acid residues and has six transmembrane domains. Decreasing ambient temperature activates the opening of a gate in the transmembrane loops and non-specific cation entry into the cell. The depolarization of sensory neurons transmits signals to the brain primarily via Aδ (and some C) fibers. While this physiological role of TRPM8 is valid for physical changes in temperature, translation of this molecular event to practical applications is more complex.

Menthol is a prototype TRPM8 “cooling” agent, but it is a multivalent ligand. Menthol stimulates TRPM8 and TRPV3, a receptor associated with warmth [Macpherson et al., More than cool: promiscuous relationships of menthol and other sensory compounds. Mol Cell Neurosci 2006; 32:335-343, 2006]. Menthol potently stimulates TRPA1 and inhibits it at higher concentrations [Karashima Y. et al. Bimodal action of menthol on, the transient receptor potential channel TRPA1. J Neurosci. 2007; 27(37):9874-84]. Thus, menthol effects are hard to interpret.

Using the “cool” of a cooling agent to treat upper gastrointestinal distress and the “burn” of heartburn is plausible. For example, Zanone (U.S. Pat. No. 6,497,859) suggested that p-menthanecarboxamides combined with menthyl acetate and a solubilizer could be useful. Bancovin et al. had interesting experimental results [The infusion of menthol into the esophagus evokes cold sensations in healthy subjects but induces heartburn in patients with gastroesophageal reflux disease (GERD). Dis. Esophagus. 2019; 32(11):1-6]. He stated, “We hypothesized that the infusion of the TRPM8 activator menthol into the esophagus leads to cold sensations and may alleviate heartburn. Surprisingly, we found that although menthol evoked the expected cold sensations from the esophagus in healthy subjects, it was also effective in causing substantial heartburn in patients with GERD.” That is, opposite to expectations, menthol evoked heartburn in GERD patients! These results fall into the category of a “non-fact” because menthol is mutlivalent.

As shown in Study 4, the EC₅₀ [median effective dose] of a candidate for activating TRPM8 has little predictive value in identifying a candidate for treating sensory discomfort in the esophageal tract. To over-emphasize the EC₅₀ value is somewhat naïve. The 95% Confidence Limits of many EC₅₀ values overlap each other. The EC₅₀ values do not give information on the quality of the heat abstraction sensation, the duration of action, or the likelihood of unpleasant taste. Thus, the choice of agents requires specific bioassays and an optimized delivery system.

When it became clear that TRPM8 receptor potency screening was mediocre as a primary method of selecting an active ingredient, it became essential to define the criteria for choosing a test compound. These criteria appear below. Any compound may overlap in activity, but usually, one compound has unique features that determine its acceptability for use.

### Criteria for Selection of an Active Ingredient

There is a general acceptance that the ion channel TRPM8 is the principal physiological element that transduces to the brain the cooling effects of agents such as menthol and icilin [McKemy et al., Identification of a cold receptor reveal a general role for Trp channels in thermosensation, Nature, 416, 52-58, 2002]. TRPM8 is a protein with 1104-amino acid residues and has six transmembrane domains. Decreasing ambient temperature activates the opening of a gate in the transmembrane loops and non-specific cation entry into the cell. The depolarization of sensory neurons transmits signals to the brain primarily via A6 (and some C) fibers. While this physiological role of TRPM8 is valid for physical changes in temperature, translation of this molecular event to practical applications is more complex.

Menthol is a prototype TRPM8 “cooling” agent, but it is a multivalent ligand. Menthol stimulates TRPM8 and TRPV3, a receptor associated with warmth [Macpherson et al., More than cool: promiscuous relationships of menthol and other sensory compounds. Mol Cell Neurosci 2006; 32:335-343, 2006]. Menthol potently stimulates TRPA1 and inhibits it at higher concentrations [Karashima Y. et al. Bimodal action of menthol on the transient receptor potential channel TRPA1. J Neurosci. 2007; 27(37):9874-84]. Thus, menthol effects are hard to interpret.

Using the “cool” of a cooling agent to treat upper gastrointestinal distress and the “burn” of heartburn is plausible. For example, Zanone (U.S. Pat. No. 6,497,859) suggested that p-menthanecarboxamides combined with menthyl acetate and a solubilizer could be useful. Bancovin et al. had interesting experimental results [The infusion of menthol into the esophagus evokes cold sensations in healthy subjects but induces heartburn in patients with gastroesophageal reflux disease (GERD). Dis. Esophagus. 2019; 32(11):1-6]. He stated, “We hypothesized that the infusion of the TRPM8 activator menthol into the esophagus leads to cold sensations and may alleviate heartburn. Surprisingly, we found that although menthol evoked the expected cold sensations from the esophagus in healthy subjects, it was also effective in causing substantial heartburn in patients with GERD.” That is, opposite to expectations, menthol evoked heartburn in GERD patients! These results fall into the category of a “non-fact” because menthol is mutlivalent.

As shown in Study 4, the EC₅₀ [median effective dose] of a candidate for activating TRPM8 has little predictive value in identifying a candidate for treating sensory discomfort in the esophageal tract. To over-emphasize the EC₅₀ value is somewhat naïve. The 95% Confidence Limits of many EC₅₀ values overlap each other. The EC₅₀ values do not give information on the quality of the heat abstraction sensation, the duration of action, or the likelihood of unpleasant taste. Thus, the choice of agents requires specific bioassays and an optimized delivery system.

When it became clear that TRPM8 receptor potency screening was mediocre as a primary method of selecting an active ingredient, it became essential to define the criteria for choosing a test compound. These criteria appear below. Any compound may overlap in activity, but usually, one compound has unique features that determine its acceptability for use.

### Shaped-Gel Design for Delivery to the Hypopharyngeal-Esophageal Target

The movement of solids and liquids from the mouth to the stomach is complicated. Matsuo et al. clearly describe the process [Anatomy and Physiology of Feeding and Swallowing: Normal and Abnormal. Phys Med Rehabil Clin N Am. 2008:19(4):691-707] and their paper is incorporated herein by reference. Cook et al. [Opening mechanisms of the human upper esophageal sphincter. Am J Physiol. Gastrointest Liver Physiol. 1989; 257, G749-G759] analyzed events during the oral and pharyngeal phases of swallowing a 2 to 10 mL barium bolus. Initially, the bolus is in the anterior mouth with the tongue tip positioned against the upper incisors. The oral cavity seals off the oropharynx by compressing the palate against the tongue. The sphincter diameter increases linearly when bolus volume goes from 2 to 10 mL but does not change further at 30 mL. The elliptical sphincter area increases from ˜120 mm² to about ˜240 mm² when bolus size increases. The midline diameter of the sphincter is only ˜5 mm. Thus, trapping and prolonging the residence time of a Shaped-Gel is feasible, especially if the Shaped-Gel dissolves and releases its contents in the pharynx.

The efficient swallowing mechanisms, exemplified by the PTT of ≤1 sec, emphasize how swallowing is biologically optimized to avoid the entry of solid particles into the airway and allow the fast entrance of food or liquids into the stomach. The epiglottis, like a trapdoor, closes and re-opens within seconds of swallowing, as does the upper esophageal sphincter. Usually, a standard softgel ensures a rapid passage from the pharynx to the stomach. The gelatin coating and the liquid that accompanies the swallow enhance the lubricity of the capsule. For a small softgel, e.g., ≤325 mg, wetting of the capsule by saliva may be sufficient for swallowing. However, for larger capsules of up to 1 g, a sip of liquid may facilitate swallowing, ≥2 mL of water.

This invention aims to shape the Shaped-Gel to retard its transit time and “trap” it in the valleculae and pyriform sinus above the upper esophageal sphincter. The Shaped-Gel shape is such that its contents release and uniformly disperse in saliva and adheres to receptors in the hypopharynx and esophagus. This strategy is counter-intuitive to standard formulations, which shape the Shaped-Gel to improve swallowability and transit to the stomach. The criteria for an optimized Shaped-Gel are:
A mass of 0.3 to 1 g, with a preferred mass of 0.4 to 0.6 g. A ≤0.3 g Shaped-Gel may transit too quickly past the upper esophageal sphincter and not have time to dissolve. A ≥1 g Shaped-Gel is challenging to swallow for many individuals.
Some individuals may need instructions to swallow the Shaped-Gel with a volume of ≤2 mL of liquid. Instructions to sip from a plastic bottle of water is also acceptable (volumes of 3 to 5 mL). Larger volumes of swallowed liquid will wash the Shaped-Gel too quickly into the lower esophagus.
The Shaped-Gel should have a flat shape such that the shortest axis of the object is 5 to 45% the length of the longest axis. This flat shape facilitates the placement of the Shaped-Gel on the tongue's surface and glides it into the pharynx. The flat shape also increases the surface area of the Shaped-Gel.
The Shaped-Gel, can have cut-outs. e.g., from the center to facilitate the dissolution of the Shaped-Gel in saliva. The cut-out in a toroid Shaped-Gel is equivalent to the “donut hole.” Punch holes can be made in a rectangular prism. A hemitoroid Shaped-Gel also works, especially for trapping in the pyriform sinuses. A pentagon Shaped-Gel was tested, but its shape may be too foreign to patients for general use.
The Shaped-Gel is made with a gelatinous matrix such that the final product is soluble in water or saliva. Thus, if the Shaped-Gel is aspirated into the airway, it is still safe to use because it will dissolve and be absorbed.
The Shaped-Gel is made with a gelatinous matrix, a plasticizer such as glycerol or sorbitol, and water. The final product is stable to store and package (e.g. blister packs). The Shaped-Gel may be further adapted for use by special populations, e.g., young children, the elderly, and disabled individuals with difficulties in salivating or swallowing.

The agent's delivery schedule may be as a fixed-interval drug or on an “as-needed” basis by the patient. Through this therapeutic regimen, the individual has voluntary control of pharyngeal-esophageal discomfort, enabling better sleep, peace of mind, and less anxiety. A fixed interval regimen may work well for treating chronic refractory cough wherein the goal is to reduce neuronal hypersensitivity.

### Mensuration of Shaped-Gel Formulation for Delivery

All three-dimensional objects have a surface area and a volume. The sphere has the lowest surface area/volume ratio, and the Menger Sponge can have an infinite surface area. Between these extremes is the optimal Shaped-Gel shape for swallowing and delivering a cooling agent to the hypopharyngeal-esophageal surface. Swallowing a solid of ≥0.6 g may be complex for some subjects. A Shaped-Gel, even with good lubricity, becomes difficult to swallow at ≥0.8 g. Therefore, a preferred mass of the Shaped-Gel is 0.3 to 0.6 g. A flat shape is easier to swallow from the tongue because the tablet can glide into the back of the throat. A flat shape is better than a sphere, an ovoid, or an oblong shape because such shapes get swallowed too quickly past the esophageal sphincter.

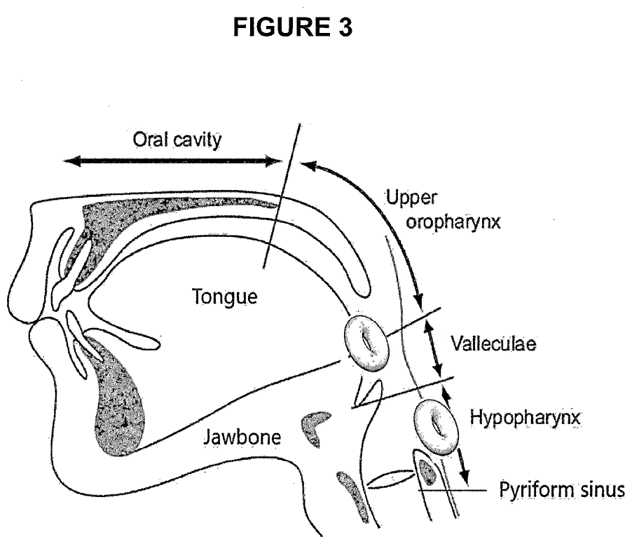

A Shaped-Gel shape with a greater surface area dissolves better in saliva. The active ingredient releases onto the TRPM8 receptors on basal epithelial layers of the foodway. Placing cavities or “holes” in the tablet increases surface area, but stability for packaging and storage limits the number of punchable holes. A toroid shape achieves a simple increase in surface area. Punching holes increases surface area and facilitates dissolution for a rectangular prism. If the number of cavities is excessive, the surface tension in the cavity, created by the saliva's mucous proteins, may hinder the tablet's dissolution. FIG. 3. shows a toroid Shaped-Gel delivered to the valleculae and pyriform sinus, as an illustration of the practice of this invention. The delivery unit is a gelatin-glycerol-water mixture having a 0.3 to 0.6 g mass.

A torus (donut-shape) is a 3-dimensional surface generated by rotating a circle of radius r around an axis within the circle's plane. The distance between the axis and the circle center is known as the major radius (R), whereas the circle radius is called the minor radius (r). The surface area and volume are given by these equations: Surface Area=4πRr, Volume=2π²Rr². For a Shaped-Gel with an R=0.9 cm and an r=0.09 cm, the Surface Area to Volume ratio is 22. For a sphere, the Surface Area to Volume is 3. Thus, dissolution of a toroid Shaped-Gel is more likely than a spherical Shaped-Gel. Planar sides can increase the surface area on the Shaped-Gel, but such shapes may not be patient-friendly. Shaped-Gels shaped to increase surface area and contemplated in this discovery are cones, cubes, cylindrical tanks, rectangular tanks, capsules, caps, conical frustums, pentagons, ellipsoids, and square pyramids.

The gelling agent employed includes but is not limited to gelatin, agar, algin, carrageenan, guar gum, gum arabic, locust bean gum, pectin, and modified starch, and mixtures thereof. In a preferred embodiment of the present disclosure, the gelling agent used is gelatin. The plasticizer is glycerol as one of the preferred embodiments of the present disclosure. The plasticizer mixes with the gelling agent and a smaller amount of water. A releasing agent such as lecithin, oil, starch, or vegetable oil, may be used to release or lubricate the gel from adhering to its dispensing and manufacturing system. The purpose of the releasing agent is also to help eject the Shaped-Gel from preformed cavities of the blister pack. Other agents, such as sweetening agents (e.g., sucralose, aspartame) or flavoring agents, may be used to enhance the perception of the Shaped-Gel (e.g., coloring agents). Other pharmaceutically acceptable excipients include diluents, disintegrants, binders, surfactants, emulsifiers, and the like.

Agonist Potency and Selectivity on TRP channels: TRPM8, TRPV1, and TRPA1

In the first set of data, the potency and in vitro effects of test compounds were evaluated on cloned hTRPM8 channel (encoded by the human TRPM8 gene, expressed in CHO cells) using a Fluo-8 calcium kit and a Fluorescence Imaging Plate Reader (FLIPR^{TETRATM}) instrument. The assays were by ChanTest Corporation (Cleveland, Ohio 44128, USA). Test solutions were in HEPES-buffered saline, 384-well plates, and placed into the FLIPR instrument (Molecular Devices Corporation, Union City, Calif., USA). Four 4 to 8 concentrations were tested, with L-menthol as the positive control. The test cells were Chinese Hamster Ovary (CHO) cells stably transfected with human TRPM8 cDNAs. The concentration-response data were analyzed via FLIPR Control software and fitted to a Hill equation for the EC₅₀. The 95% Confidence Interval was from GraphPad Prism 6 software.

Table 5 summarizes the agonist activity in the TRPM8 receptor assay. All tested compounds showed full efficacy, i.e., at the highest tested concentration, there was ˜100% stimulation of calcium entry, and the data fitted a sigmoidal dose-response curve. The EC₅₀ of the more potent sensory compounds DIPA-1-6 to 1-9 and DIPA-2-5 to 2-8 fell within a narrow range with overlapping 95% Confidence Intervals. No distinguishing features in the EC₅₀ predicted compounds with desired cooling properties in the esophageal tract. The structural modifications of 3-1 and 3-2 resulted in a significant loss of bioactivity.

Selectivity was studied using cells transfected with TRPM8, TRPV1 channels (human TRPV1 gene expressed in HEK293 cells), and TRPA1 channels (human TRPA1 gene expressed in CHO cells). The selectivity of DIPA-1-9 on TRP channel receptors, TRPM8, TRPA1, and TRPV1 is shown in FIG. 3 of Yang et al. A novel TRPM8 agonist relieves dry eye discomfort. BMC Ophthalmology (21017) 17: 101, and incorporated herein by reference. The applicant is a co-author of this publication. Selectivity is also seen with DIPA-1-7 and DIPA-1-8 (data in FIG. 1, Wei U.S. Pat. No. 9,956,232). These test cells were also from Chinese Hamster Ovary (CHO) cells or Human Embryonic Kidney (HEK) 293 cells transfected with human TRPV1 or TRPA1 cDNAs. The positive control reference compound was capsaicin (a known TRPV1 agonist) or mustard oil (TRPA1 agonist).

In summary, the relative potencies of these test series, as measured by the TRPM8 EC₅₀ [median effective dose], seem to have limited predictive value for comparisons. The 95% Confidence Limits of many EC₅₀ overlap, and only analogs with at least a 5-fold difference in potency are distinguishable. The choice of an ideal ingredient requires getting the right degrees of coolness and avoiding icy cold sensations and adverse tastes. Furthermore, the duration of action is an important parameter. However, the EC₅₀ does not give information on the quality of the heat abstraction sensation, the likelihood of unpleasant taste, or the duration of the drug effect. Thus, the EC50 is insufficient to define the desirable drug actions (access to and efficacy at TRPM8). To over-interpret the EC₅₀ is naïve. Other bioassays are required to address the questions of selectivity and specificity. The 3,4-6 and 3,4-7 analogs described as the most active in '496 had weak TRPM8 potencies.

Study 1

General Considerations

In initial feasibility studies, three methods of applying a cooling agent to the throat were compared: namely, with a sprayer, by drops, or by a swallowed gel (Table 6). The dosimetry was such that a gel could deliver the largest dose without adverse events like bad taste or icy cold. The gel method gave a quantum jump in the duration of action, which was a major advantage. Cooling was felt at Adam's apple with all three delivery methods, but the gel gave the best results for the upper chest. Thus, the cooling agent in the gel gets onto the esophageal surface. Spray and drops were more likely to get into the mouth and affect the taste, a disadvantage not seen with the gel. The spray droplets could also get into the airways and trigger cough, a risk not seen with the drops or gel. The spray is a patient-familiar method of drug delivery, the drops less so, and swallowing the gel requires patient instruction. But patients soon become used to the gel, which has distinct advantages. If one weighs the evidence in toto, the gel is an excellent method for drug delivery to the pharynx and esophagus and is superior to spray or drops. Surprisingly, this method of gel delivery has not been much utilized for esophageal drug delivery.

TABLE 5
TRPM8 agonist activity of test compounds.

| | EC₅₀ | 95% Confidence | Relative Potency |
|---|---|---|---|
| Compound | (μM) | Interval | to L-menthol |
| Menthol | 3.8 | 2.5 to 5.6 | 1.0 |
| DIPA-1-5 | 5.6 | 4.4 to 7.2 | 0.7 |
| DIPA-1-6 | 2.4 | 1.5 to 4.0 | 1.6 |
| DIPA-1-7 | 0.7 | 0.5 to 1.0 | 5.4 |
| DIPA-1-8 | 0.7 | 0.5 to 1.0 | 5.4 |
| DIPA-1-9 | 0.9 | 0.4 TO 2.5 | 4.0 |
| DAPA-2-4 | 14.5 | 7 to 29 | 0.3 |
| DAPA-2-5 | 1.7 | 1.0 to 2.9 | 2.2 |
| DAPA-2-6 | 0.8 | 0.5 to 1.3 | 4.7 |
| DAPA-2-7 | 1.1 | 0.6 to 2.3 | 3.4 |
| DAPA-2-8 | 1.3 | 0.7 to 2.3 | 2.9 |
| DAPA-3-1 | 24 | 8 to 76 | 0.2 |
| DAPA-3-2 | 4.2 | 1.6 to 10.8 | 0.9 |

TABLE 6
Delivery Systems to Surfaces of Pharynx-Esophagus

| Parameter | Sprayer | Drops | Gel | Comments |
|---|---|---|---|---|
| volume, mass | 0.4 mL | 0.4 mL | 0.3 to 0.8 g | — |
| concentration (mg) | 3 | 10 | 10 | — |
| total dose (mg) | ~1.2 | ~4 | ~3 to 8 | gel advantage |
| duration | ~10-15 | ~30-45 + | 60 + | gel advantage |
| mouth | +++ | ++ | 0 | gel advantage |
| upper throat | +++ | +++ | ++ | target jugular notch |
| chest | 0 | + | +++ | gel advantage |
| adverse taste | + | + | 0 | gel advantage |
| risk of entry to lung | + | 0 | 0 | — |
| familiarity of use | +++ | ++ | + | — |

### Study 2

### Sensory Qualities of Compounds Applied to Oral Cavity and Pharynx

Tests were on four volunteers, with 3 to 5 trials per substance. Compounds were prepared in cherry-flavored Shaped-Gel at 5 mg/mL and administered ˜0.8 mL per dose with a 2 mL plastic vial to the base of the tongue. The subjects asked to rate the sensations for cooling intensity, cold discomfort and adverse taste. Surprisingly, the sensory results were clearcut and there were no ambiguities about the sensory effects that were elicited. The compounds DIPA-1-7, DIPA-1-8, DAPA-2-6, DAPA-2-7 and 3,4-7 produced cold, icy cold, and adverse tastes which were instantly recognized and disliked. In particular, DIPA-1-7 produced icy pain in the back of the throat and was considered aversive. 3,4-6 produced robust cooling, but its duration of action 5 to 10 min, were too short to be of therapeutic value. By contrast, DIPA-1-9 Shaped-Gel produced a coolness and cold which was well-tolerated and the concentration could be increased to 15 mg/mL without objections: that is, there was no pain or discomfort.

The unpleasant tastes produced by DIPA-1-7, DIPA-1-8, 2-6, 2-7, and 2-8 were described as “metallic”, “organic solvent-like”, and “harsh” which lasted for at least 15 min. The subjects said these taste qualities were unpleasant and undesirable. When tested in the evening near sleep time, the perception of cooling in the throat was more pronounced presumably because there were fewer environmental cues for distraction. In these situations, the heat abstraction sensations were perceived for ≥20 min. Although overt cooling sensation may not be felt after 15 min, the general sense of refreshment in the throat from DIPA-1-9 may persist for 3+ hours. Surprisingly, increasing the test concentration of DIPA-1-9 from 5, to 8 to 15 mg/mL in simple Shaped-Gel and a volume of 0.8 mL per dose did not produce icy cold or pain. Thus, there is a safety margin in the use of DIPA-1-9 without risks of a painful throat.

The unexpected observation here was DIPA-1-9 has good qualities of cooling sensation. But in the other analogs, where the alkyl chain is n-hexyl, n-heptyl, or n-octyl, the chemicals cause cold discomfort and adverse taste (FIG. 7). Furthermore, the duration of action DIPA-1-9 was sufficiently long to be of clinical value. Thus, these trials showed, surprisingly, that DIPA-1-9 is uniquely the best ingredient for sensory discomfort in the pharynx. The qualitative differences in DIPA-1-9 that makes it selective and exceptional could not have been predicted from prior art. It was concluded that DIPA-1-9 is the best candidate as an antinociceptive agent for the esophageal tract.

The 1-di-sec-butyl-phosphorylpentane (DAPA-8) was also tested in Shaped-Gel, but its duration of action was too short to be of practical value. Like 3,4-6 its duration of action was about 5 to 10 min. It is possible that DIPA-1-8 will be a better agent than DIPA1-9 for situations where there is excess exudate (mucus and phlegm) in the trachea, larynx, pharynx, and eosphagus, because DIPA-1-8 can more easily reach the TRPM8 receptors in stratum basale than DIPA-1-9.

### Study 3

Shaped-Gel having total weight of 0.4 to 1.2 g containing 0.4 to 1.5% by weight (5 mg/g to 15 mg/g) are prepared and tested. The following examples illustrate the invention, but are not intended to limit the scope of the present invention.
A Shaped-Gel in accordance with the present invention was prepared with the following composition.

### Each SHAPED-GEL: ˜1 g

### Cooling Agent: DIPA-1-9, 10 mg (1%)

### Gelatin: 120 mg (12%)

### Glycerol: 650 mg (65%)

### Water: 220 mg (22%)

### Each SHAPED-GEL: ˜0.8 g

### Cooling Agent: DIPA-1-9, 8 mg (1%)

### Gelatin: 96 mg (12%)

### Glycerol: 520 mg (65%)

### Water: 176 mg (22%)

### Each SHAPED-GEL: ˜0.8 g

### Cooling Agent: DIPA-1-7, DIPA-1-8, 8 mg (1%)

### Gelatin: 96 mg (12%)

### Glycerol: 520 mg (65%)

### Water: 176 mg (22%)

### Each SHAPED-GEL: ˜0.8 g

### Cooling Agent: DAPA-2-5, DAPA-2-6, 8 mg (1%)

### Gelatin: 96 mg (12%)

### Glycerol: 520 mg (65%)

### Water: 176 mg (22%)

### Each SHAPED-GEL: ˜1.0 g

### Cooling Agent: Ax-8 10 mg (1%), WS-5, WS-12, or WS-30

### Ethanol: 50 mg (5%) g

### Gelatin: 120 mg (12%)

### Glycerol: 650 mg (62%)

### Water: 220 mg (20%)

Shaped-Gels were prepared by these procedures. First step, the cooling agent was accurately weighed and place in a 60 mL plastic container. Then glycerol, water, and gelatin was added to the container and centrifuged for 2 min in a SpeedMixer (FlackTek, Inc.) at 3000 rpm. Then gelatin powder was added and the mixture centrifuged for 3 min. The container was warmed by placing it in a beaker containing hot water (80-85° C.) for 15 min. The container was then centrifuged for 5 min. The liquid mix was cooled and dispensed onto silicon molds with a plastic dropper, and placed in a refrigerator. After one hour, the gels were solidified, removed from the mold with a plastic tweezer, weighed and placed on wax paper and later stored in blister pockets. For some active ingredients 5% ethanol was added to increase solubility.
In preliminary studies, the shape of the Shaped-Gel was varied. Square, rectangular, spherical, hemispherical, toroidal, and pentagram Shaped-Gels were prepared at sizes ranging from 0.4 to 1.2 g. A flat shape wherein the shortest diameter was about 20% of the longest axis was preferred because it could be placed flat on the back of the tongue. A Shaped-Gel shaped as a rectangular prism of 2.75 mm×15 mm×15 mm=0.63 cm3 and weighing ˜0.6 g was acceptable to subjects. A toroid, much like a “LiveSaver” mint was also acceptable if the size was ˜0.8 g.
Test Panel: The volunteers for testing the Shaped-Gel were six subjects, four males and two females, aged 50 to 76 years. Each Shaped-Gel was tested for at least four trials in at least three subjects. The concentration of the cooling agent in the Shaped-Gel varied from 0.5 to 2%. The duration of observation for each trial usually lasted for not more than 1.5 hours. Subjects were instructed to swallow the Shaped-Gel with a sip of room temperature cold water. Cooling sensations were rated on a scale of 0, 1, 2, 3, at intervals of 3 min, 5 min, and then at 10 min intervals. If a cooling value exceeded 2, the subject noted whether the cooling was felt at the level of the Adam's apple, jugular notch or upper chest. Ancillary effects were also recorded.

TABLE 7
Testing of Various Cooling Agents in a Shaped-Gel.

| | | Duraton | Adam’s | Jugular | | |
|---|---|---|---|---|---|---|
| Ingredient | Onset | min | Apple | Notch | Sternum | Discomfort |
| DIPA-1-9 | <3 | ~60 | ++ | ++ | ++ | 0 |
| DAPA-2-6 | <3 | 30 | +++ icy | ++ icy | ++ | yes |
| Ax-8 | 5 | 35 | ++ | + | + | yes |
| WS-5 | ~3 | 25 | ++ | + | + | 0 |
| WS-12 | 5 | 20 | + | 0 | 0 | 0 |
| WS-30 | ~3 | 25 | ++ | + | + | 0 |

These general effects were consistently observed: a rapid onset 3 min) of the sensation of coolness in the throat after application of DIPA-1-9 Shaped-Gel to the base of the tongue. The coolness spreads to the rest of throat and intensifies, as if a spoonful of rich ice cream had been swallowed. This cooling effect lasts for 45 min to up to 3 hr, and any prior discomfort in the throat is relieved. The cooling sensation can be used to facilitate mucus expectoration from the airways. Also relieved is the sense of suffocation when lying down to sleep in a subject that has dyspnea. Other cooling agent were also examined, as shown in the Table 7 and 8.

TABLE 8
Structures of p-Menthane compounds tested in Shaped-Gel.

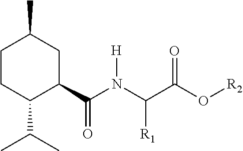

| | | | Philtrum | Eyelids | | Potency |
|---|---|---|---|---|---|---|
| | | | Skin | Surfaces | | relative to |
| Compounds | R₁ | R₂ | (min) | (min) | EC₅₀ μM | menthol |
| Menthol | | | | | 3.8 | 1.0 |
| | | | | | (2.5 to 5.6) | |
| Gly-OEt (WS- | H | Et | 24 | 15 | 0.36 | 10.6 |
| 5) | | | | | (0.30 to 0.75) | |
| Gly-OiPr-(Ax- | H | iPr | 27 | 300 | 0.50 | 7.6 |
| 8) | | | | | (0.27 to 0.85) | |
| D-Ala-OEt | Me | Et | 103 | 180 | 0.270 | 14.0 |
| | | | | | (0.17 to 0.42) | |
| D-Ala-OiPr | Me | iPr | 34 | 360 | 0.260 | 14.6 |
| | | | | | (0.16 to 0.41) | |

In summary, the idea has been put forward to deliver, by topical application a molecule, to the hypopharynx and upper esophagus in such a fashion that the delivery system is intercepted in the lumens of the hypopharynx and eosphagus. This delay in transit then allows more time for the active ingredient receptors located in the hypopharynx and esophagus. The active ingredient has evokes a cooling sensation the central nervous system which can alleviate discomforts of the esophagus. Specifically, the symptoms relieved are heartburn, epigastric pain, discomfort of regurgitation, a sense of bloat, a sense of indigested food, dyspepsia, belching and the like. By synthesizing compounds called 1-dialkyl-phosphinoyl-alkanes (DAPA) and devising tests on them, two molecules named DIPA-1-8 and DIPA-1-9 were identified as having the selective properties for achieving the desired sensory effect: namely, an ideal cooling of the throat at the level of the Adam's Apple, jugular notch and manubrium. The receptor target was selective for TRPM8 and not TRPVI and TRPA1. The DIPA were formulated in a Shaped-Gel matrix at a mass of 05 to 0.8 g and having a flat surface that facilitated swallowing. The dose of DIPA per Shaped-Gel was 3 to 15 mg and effective for reducing sensory discomfort of the esophageal tract in a subject in need of treatment.

1. A therapeutic method to treat symptoms of a disorder of the esophagus in a person in need thereof, comprising:
orally administering a medicament that contains a cooling agent in a gelatin-glycerol-water matrix, said cooling agent consisting essentially of a 1-dialkyl-phosphinoyl-alkane, wherein said medicament weighs from 0.3 to 1.0 g per unit dose,
and has a flat shape with a shortest axis that is 5 to 45% of its longest axis,

2. The method as in claim 1 wherein the cooling agent comprises at 0.4% (5 mg/g) to 1.5% (15 mg/g) by weight of the medicament.

3. The method as in claim 1 wherein the medicament has a shape of a rectangular prism.

4. The method as in claim 1 wherein the medicament has a shape of a toroid.

5. The method as in claim 1 wherein the medicament has one or more holes in the medicament matrix.

6. The method as in claim 1 wherein the glycerol-gelatin matrix has a weight ratio of glycerol to gelatin that is at least two.

7. The method as in claim 1 wherein the gelatin-glycerol-water matrix of the medicament pill is comprised of a gelatin of animal, vegetable, or marine origin.

8. The method as in claim 1 wherein the cooling agent is 1-Diisopropyl-phosphinoyl-octane, referred to herein as DIPA-1-8.

9. The method as in claim 1 wherein the cooling agent is 1-Diisopropyl-phosphinoyl-nonane, referred to herein as DIPA-1-9.

10. The method as in claim 1 wherein a symptom of the disorder of the esophagus is caused by the disorder known as acid reflux, gastroesophageal reflux disease, laryngopharyngeal reflux, dyspepsia, or indigestion.

11. The method as in claim 1 wherein a symptom of the disorder of the esophagus is heartburn.

12. The method as in claim 1 wherein a symptom of the disorder of the esophagus is epigastric pain or chest pain.

13. The method as in claim 1 wherein a symptom of the disorder of the esophagus is non-cardiac chest pain.

14. The method as in claim 1 wherein a symptom of the disorder of the esophagus is sensation of fullness, bloat, belching, or a sour taste in the throat.

15. The method as in claim 1 wherein a symptom of the disorder of the esophagus is the discomfort from abstaining from smoking a mentholated cigarette.

16. The method as in claim 1 wherein the medicament is shaped as a flat square with three holes arranged triangularly in the middle of the tablet.

17. The method as in claim 1 wherein the medicament is shaped as a flat torus.

18. The method as in claim 1 wherein the medicament is packaged in a form of a single or a multiple dose.

19. A therapeutic method to treat symptoms of a disorder of the esophagus in a person in need thereof, said therapeutic method comprising:
providing a medicament being adapted for oral administration with delayed transit time in the esophagus, said medicament comprising a 1-dialkyl-phosphinoyl-alkane effective as a cooling agent and being carried in a gelatin-glycerol-water matrix, wherein said medicament weighs from 0.3 to 1.0 g per unit dose and said medicament has a flat shape with a shortest axis that is 5 to 35% of its longest axis.

20. The method as in claim 19 wherein the 1-dialkyl-phosphinoyl-alkane is 1-Diisopropyl-phosphinoyl-octane or 1-Diisopropyl-phosphinoyl-nonane.

21. The method as in claim 19 wherein a symptom of the disorder of the esophagus is caused by the disorder known as acid reflux, gastroesophageal reflux disease, laryngopharyngeal reflux, dyspepsia, or indigestion.
